(19) United States
(12) Patent Application Publication
(10) Pub. No.: US 2024/0009170 A1
(43) Pub. Date: Jan. 11, 2024

(54) INHIBITION OF GLYCOGEN SYNTHASE KINASE-3 (GSK-3)

(71) Applicant: Brown University, Providence, RI (US)

(72) Inventors: Wafik S. EL-DEIRY, Barrington, RI (US); Kelsey HUNTINGTON, Providence, RI (US); Benedito A. CARNEIRO, East Greenwich, RI (US)

(21) Appl. No.: 18/251,868

(22) PCT Filed: Nov. 17, 2021
(86) PCT No.: PCT/US2021/059771

§ 371 Date: May 4, 2023

Related U.S. Application Data
(60) Provisional application No. 63/115,879, filed on Nov. 19, 2020.
(60) Provisional application No. 63/114,787, filed on Nov. 17, 2020.

(51) Int. Cl. A61K 31/404; A61K 33/14; A61K 45/06; A61P 35/00

(52) U.S. Cl. CPC A61K 31/404; A61K 33/14; A61K 45/06; A61P 35/00

(57) ABSTRACT

The invention provides small-molecule inhibition of GSK-3 to increase efficacy of ICB and improve response in patients with microsatellite stable colorectal cancer, and possibly other tumor types. These results demonstrate 9-ING-41 in combination with αPD-1/PD-L1 therapy.

### STATEMENT REGARDING FEDERALLY SPONSORED RESEARCH OR DEVELOPMENT

This invention was made with government support under grant number CA173453 awarded by National Institutes of Health. The government has certain rights 20 in the invention.

### TECHNICAL FIELD OF THE INVENTION

This invention generally relates to heterocyclic compounds containing both one or more hetero rings having oxygen atoms as the only ring hetero atoms, and one or more rings having nitrogen as the only ring hetero atom containing three or more hetero rings, and specifically to GSK-3 inhibitors belong to a class of maleimide derivatives containing a maleimide group.

### REFERENCE TO RELATED APPLICATIONS

This invention is related to provisional patent applications U.S. Ser. No. 63/114,787, filed Nov. 17, 2020, and U.S. Ser. No. 63/115,879, filed Nov. 19, 2020.

### BACKGROUND OF THE INVENTION

Colorectal cancer (CRC) represents about one in ten cancer cases. Sung et al., CA Cancer J. Clin., 71, 209-249 (2021). Globally, colorectal cancer ranks third in terms of incidence and second in terms of mortality. Colorectal cancer treatment options include surgery, chemotherapy, radiation therapy, targeted therapy, and immunotherapy.

Immune checkpoint blockade (ICB) is now being used in clinical care for colorectal cancer. The United States Food & Drug Administration approved the checkpoint inhibitors nivolumab® and pembrolizumab® for treating microsatellite instability-high (MSI-H) colorectal cancer after chemotherapy. Immune checkpoint blockade clinical trials demonstrated efficacy in microsatellite instability-high colorectal cancer. The impressive durability of tumor regression contrasts starkly with the lack of response observed in microsatellite stable (MSS) colorectal cancer. Thus, there remains in the oncological art a substantial unmet need to treat the −85% of patients with microsatellite stable colorectal cancer, for whom immune checkpoint blockade is less effective. See Gupta, Sinha, & Paul, Current Problems in Cancer, 42, 548-559 (2018).

Recently, a growing body of literature is characterizing the immunomodulatory function of glycogen synthase kindase-3 (GSK-3) in the context of anti-tumor immunity. See Augello et al., Cells, 9 (2020). GSK-3 inhibition inhibits the transcription of inhibitory co-receptor LAG-3 for enhanced anti-tumor immunity and compensates for the lack of CD28 in the priming of CD8+ T cells. Rudd, Chanthong, & Taylor, Cell Reports, 30, 2075-2082.e2074 (2020). The rescue of exhausted CD8+ cytolytic T cells by αPD-1 blockade has been found to require CD28 expression and GSK-3 is the central co-signal for CD28 priming of CD8+ T cells in αPD-1 immunotherapy, which has implications in cancers with low CD28 expression. Moeller et al., Cancer Gene Ther., 11, 371-379 (2004). GSK-3 also controls T cell motility and induces proteasomal degradation of PD-L1 in tumor cells. Taylor & Rudd, BMC Research Notes, 13, 163-163 (2020).

The GSK-311/11-catenin axis activates vascular endothelial growth factor (VEGF) signaling in endothelial cells to promote angiogenesis. VEGF is immunosuppressive, dampening the immune cell response by promoting recruitment of tumor-associated macrophages (TAMs). Thus, VEGF may be a target to modulate antitumor immunity and tumor metastasis.

Immune checkpoint blockade (ICB) has demonstrated efficacy in treating microsatellite instability positive (MSI+) colorectal cancer. However, there remains in the oncological art a substantial unmet need to treat the approximately 96% of patients with microsatellite stable (MSS) advanced colorectal cancer who do not respond to immune checkpoint blockade.

### SUMMARY OF THE INVENTION

The invention characterizes the effects of GSK-3 inhibitors on tumor and immune cells in vitro and in combination with immune checkpoint blockade in vivo. Several GSK-3 inhibitors have structural similarity (e.g., the inclusion of the maleimide group) with 9-ING-41. The compound 9-ING-41 mediated a decrease of VEGF in conjunction with a 9-ING-41-mediated increase of BRAK secreted by the tumor cells can be used therapeutically to increase the capacity of natural killer cell-mediated and T cell-mediated killing of the tumor cells. These effects can contribute to anti-cancer activity of 9-ING-41 in cancer types.

In a first embodiment, the invention provides a method of treating colorectal cancer. The method comprises the steps of administering a compound with structural similarity (e.g., the inclusion of the maleimide group) to 9-ING-41 to a subject with colorectal cancer. The several analogs of 9-ING-41 with structural similarity can also have anti-tumor or immunomodulatory effects. A variety of formulations can be developed for these maleimide derivatives including both oral and intravenous (IV) formulations. These different formulations can be tested first in in vivo models. 9-ING-41 has already been formulated for intravenous administration in early clinical trials. “Starting dose of-9-ING-41 will be administered on Day 1 and 4 each week of a 21-day cycle. 9-ING-41 will be administered intravenously over 60 minutes.” See ClinicalTrials.gov Identifier: NCT03678883.

In a second embodiment, the invention provides a method of treating colorectal cancer, comprising the steps of administering 9-ING-41 to a subject with colorectal cancer.

In a third embodiment, the invention provides a method of treating colorectal cancer, comprising the steps of administering compound CID: 150974278 to a subject with colorectal cancer. The characteristics of this compound can be compared to 9-ING-41. See National Library of Medicine (National Center for Biotechnology Information), CID:150974278.

In a fourth embodiment, the invention provides a method of treating colorectal cancer, comprising the steps of administering compound CID: 137495982 to a subject with colorectal cancer. The characteristics of this compound can be compared to 9-ING-41. See National Library of Medicine (National Center for Biotechnology Information), CID:150974278.

In a fifth embodiment, the invention provides a method of treating colorectal cancer, comprising the steps of administering compound CID: 59140652 to a subject with colorectal cancer. The characteristics of this compound can be compared to 9-ING-41. See National Library of Medicine (National Center for Biotechnology Information), CID:150974278.

In a sixth embodiment, the invention provides a method of treating colorectal cancer, comprising the steps of administering compound CID: 16741640 to a subject with colorectal cancer. The characteristics of this compound can be compared to 9-ING-41. See National Library of Medicine (National Center for Biotechnology Information), CID:150974278.

In a seventh embodiment, the invention provides a method of using a compound such as a compound with structural similarity (e.g., the inclusion of the maleimide group) to 9-ING-41 in a therapeutic method to increase the host's anti-tumor immune response and to decrease tumor burden in conjunction with other therapeutic agents. A patient's genomic signatures and serum or plasma cytokine profiles can be useful in the prediction of response to the immune stimulatory effects of maleimide derivatives. The other anti-cancer therapeutic agent can be e.g., an immune checkpoint therapy, e.g., an αPD-1/PD-L1 therapy.

In an eighth embodiment, the invention provides a method of treating COVID-19, by administering a therapeutically effective amount of a GSK-3 inhibitor with structural similarity to 9-ING-41, wherein the GSK-3 inhibitor includes a maleimide group, to a subject with COVID-19.

In an eighth embodiment, the invention provides a method of treating COVID-19, with the additional step of administering a therapeutically effective amount of a second GSK-3 inhibitor, to a subject with COVID-19.

In a ninth embodiment, the invention provides a method of treating COVID-19, with the further step of administering a therapeutically effective amount of a second GSK-3 inhibitor, to a subject with COVID-19.

In a tenth embodiment, the second GSK-3 inhibitor is lithium.

In an eleventh embodiment, the invention provides a method of treating COVID-19, by administering a second GSK-3 inhibitor orally or sublingually. The second GSK-3 inhibitor can be lithium.

In a twelfth embodiment, the invention provides a method of treating COVID-19, comprising the additional step of monitoring the course of treatment based on inflammatory plasma or serum cytokine profiles. The profile can be e.g., high levels of C Reactive Protein (CRP).

In a thirteenth embodiment, the invention comprises the further step of monitoring the course of treatment for lithium-associated toxicity.

The results described in this specification invention demonstrate that small-molecule inhibition of GSK-3 can be a potential means to increase efficacy of immune checkpoint blockade and improve response in patients with microsatellite stable colorectal cancer, and possibly other tumor types. These findings support the therapeutic use of 9-ING-41 in combination with αPD-L1 therapy.

In a second embodiment, because of the immune modulatory effects of GSK-3 the invention provides a method of treating COVID-19. GSK-311 mediates the phosphorylation of nucleocapsid proteins important for viral replication. These effects may help antiviral agents achieve more potent disease suppression to attenuate or block COVID-19 infection. The method comprises the steps of administering to a subject with a COVID-19 infection.

Several formulations could be developed for these maleimide derivatives including both oral and intravenous (IV) formulations. Patients with early or less severe COVID19 disease may benefit from this therapeutic approach.

A patient's genomic signatures and serum or plasma cytokine profiles may be useful in the prediction of response to the anti-viral effects of maleimide derivatives.

The inventors characterized the effects of GSK-3 inhibitor 9-ING-41 in vitro and in combination with αPD-1/αPD-L1 in vivo. Inhibition of GSK-3 by 9-ING-41 led to increased natural killer and T cell-mediated colorectal cancer cell killing in a cell co-culture. Pre-treatment of tumor cells by 9-ING-41 sensitized the tumor cells to immune cell-mediated killing. Treatment of tumor cells by 9-ING-41 boosted the efficacy of αPD-1 therapy in this cellular co-culture model.

The administration of 9-ING-41 to colorectal cancer cells decreased Survivin, NF-κB p65, and IKBα expression. The administration of 9-ING-41 increased PD-L1 expression as shown via Western blot. The opposite effect occurred in immune cells, where drug treatment increased NF-κB-inducing kinase (NIK) expression. Using flow cytometric analysis of cell surface markers of tumor and immune cells treated with 9-ING-41 in vitro, the inventors observed differential expression of HLA-E, PD-L1, ICAM-1, DR5, NKG2A, TRAIL, and LAG-3.

In a syngeneic murine colon carcinoma BALB/c model using microsatellite stable cell line CT-26, the inventors observed increased survival of mice treated with 9-ING-41 alone as compared to the control group.

Historically, MSI+ patients are predicted to be more response to checkpoint blockade therapies. The MSS genotype can be because there is a greater need for improved treatment in this patient population. However, these drugs are hypothesized to act similarly in MSI+ patients.

The patient selection could be guided by genomic signatures and cytokine profiles within the MSS group. This is because most patients are MSS.

The inventors noted the highest probability of survival by Kaplan Meier curve in the 9-ING-41 and anti-PD-L1 combination therapy group, with a 50% probability of survival at 60 days post-treatment initiation (33.3% response rate), compared to 17% for the αPD-1 group (16.6% response rate), and 0% for all other treatment groups (0% response rate). When comparing T cell ratios, compared to non-responders, partial responders had a lower splenic CD8+/Treg and CD4+/Treg ratio and a higher intra-tumoral CD8+/Treg and CD4+/Treg ratio.

The inventors next used cytokine profiling on murine serum samples. They noted that complete and partial responders were more likely to have lower serum concentrations of tumorigenic cytokines BAFF, CCL7, CCL12, VEGF, VEGFR2, and CCL21 as compared to non-responders. Complete and partial responders had higher serum concentrations of immunomodulatory cytokines CCL4, TWEAK, GM-CSF, CCL22, and IL-12p70 compared to non-responders. These results demonstrate that small-molecule inhibition of GSK-3 can increase efficacy of αPD-L1 and improve response in patients with microsatellite stable colorectal cancer.

GSK-3 inhibition in combination with immune checkpoint blockade is hypothesized to be efficacious because GSK-3 inhibition can increase tumor cell PD-L1 expression, increase immune cell trafficking, and compensate for a lack of CD28 priming.

### BRIEF DESCRIPTION OF THE DRAWINGS

For illustration, some embodiments of the invention are shown in the drawings described below. Like numerals in the drawings indicate like elements throughout. The invention is not limited to the precise arrangements, dimensions, and instruments shown.

FIG. 1 is a set of three bar graphs showing cytokine, chemokine, and growth factor profiles of HCT116 colorectal cancer cell culture supernatant treated at indicated doses (IC₁₀, IC₃₀, IC₅₀, IC₇₀) for forty-eight hours. The overall cytokine, chemokine, and growth factor profiles shows a reduction with increasing doses of 9-ING-41. The bar graph in FIG. 1(A) shows the VEGF profile. The bar graph in FIG. 1(B) shows the CXCL14/BRAK profile. The bar graph in FIG. 1(C) shows the M-CSF profile.

FIG. 2 is a graph and a table showing a cell titer glo analysis to determine IC doses after treatment up to 50 μM for seventy-two hours. CellTiter-Glo was added to acquire cell viability images with the in vivo imaging system (IVIS). The graph on the left shows a normalized cell viability IC₅₀ curve for HCT116, HT29, and KM12C cell lines. The table on the right lists IC₅₀ values for indicated cell lines.

FIG. 3 is a pair of bar graphs showing that treatment of SW480 tumor cells with 9-ING-41 bolsters TALL-104 cell-mediated tumor cell killing. The inventors obtained photographic images of co-culture at 24-hour timepoint. 1:1 TALL-104 to SW480 cell ratio, 24-hour co-culture duration. EthD-1 was used to visualize dead cells, 10× magnification, scale bar indicates 100 μm. FIG. 3(A) shows the quantification of co-culture experiment using percentage of dead cells out of total cells (n=3). FIG. 3(B) shows the quantification normalized by cell death observed with drug treatment alone (n=3). A one-way Anova followed by a post-hoc Dunnett's multiple comparisons test was used to calculate statistical significance. Significance is reported as follows: P≤0.05: *, P≤0.01: **, and P≤0.001: ***.

FIG. 4 is a pair of bar graphs showing that treatment of SW480 tumor cells with 9-ING-41 bolsters CD8+ donor-derived healthy T cell-mediated cell killing. Images of co-culture at 24-hour timepoint. 1:1 CD8+ donor-derived healthy T cell to SW480 cell ratio, 24-hour co-culture duration. EthD-1 was used to visualize dead cells, 10× magnification, scale bar indicates 100 μm. FIG. 4(A) shows the quantification of co-culture experiment using percentage of dead cells out of total cells (n=3). FIG. 4(B) Quantification normalized by cell death observed with drug treatment alone (n=3). A one-way Anova followed by a post-hoc Dunnett's multiple comparisons test was used to calculate statistical significance. Significance is reported as follows: P≤0.05: *, P≤0.01: **, and P≤0.001: ***.

FIG. 5 is a pair of bar graphs showing that treatment of SW480 tumor cells with 9-ING-41 bolsters NK-92 cell-mediated cell killing. Co-culture was treated with 9-ING-41, or SW480 tumor cells were pre-treated (PT) with 9-ING-41 for twenty-four hours before the 24-hour co-culture began. Images of co-culture at 24-hour timepoint. 1:1 NK-92 to SW480 cell ratio, 24-hour co-culture duration. EthD-1 was used to visualize dead cells, 10× magnification, scale bar indicates 100 μm. FIG. 5(A) shows the quantification of co-culture experiment using percentage of dead cells out of total cells (n=3). FIG. 5(B) shows the quantification normalized by cell death observed with drug treatment alone (n=3). A one-way Anova followed by a post-hoc Dunnett's multiple comparisons test was used to calculate statistical significance. Significance is reported as follows: P≤0.05: *, P≤0.01: **, and P≤0.001: ***.

FIG. 6 is a pair of bar graphs showing that pre-treatment with 9-ING-41 sensitizes SW480 tumor cells to TALL-104 cell-mediated cell killing. Images of co-culture at 24-hour timepoint. 1:1 TALL-104 to SW480 cell ratio, 24-hour immune cell pre-treatment with 5 μM 9-ING-41, followed by 24-hour co-culture. EthD-1 was used to visualize dead cells, 10× magnification, scale bar indicates 100 μm. FIG. 6(A) shows the quantification of co-culture experiment using percentage of dead cells out of total cells (n=3). FIG. 6(B) shows the quantification normalized by cell death observed with drug treatment alone (n=3). A one-way Anova followed by a post-hoc Dunnett's multiple comparisons test was used to calculate statistical significance. Significance is reported as follows: P≤0.05: *, P≤0.01: **, and P≤0.001: ***.

FIG. 7 is a pair of bar graphs showing that pre-treatment with 9-ING-41 sensitizes SW480 tumor cells to CD8+ T cell-mediated cell killing. Images of co-culture at 24-hour timepoint. 1:1 CD8+ T cell to SW480 cell ratio, 24-hour immune cell pre-treatment with 5 μM 9-ING-41, followed by 24-hour co-culture. EthD-1 was used to visualize dead cells, 10× magnification, scale bar indicates 100 μm. FIG. 7(A) shows the quantification of co-culture experiment using percentage of dead cells out of total cells (n=3). FIG. 7(B) shows the quantification normalized by cell death observed with drug treatment alone (n=3). A one-way Anova followed by a post-hoc Dunnett's multiple comparisons test was used to calculate statistical significance. Significance is reported as follows: P≤0.05: *, P≤0.01: **, and P≤0.001: ***.

FIG. 8 is a set of four bar graphs showing that 9-ING-41 increases tumor cell death in combination with anti-PD-1 therapy in a co-culture of HCT-116 colorectal cancer cells and either TALL-104 cells or NK-92 cells. Images of a 24-hour co-culture with a 1:1 TALL-104 cell to SW480 cell ratio and 5 μM 9-ING-41+25 μg/mL nivolumab® (N) or 25 μg/mL pembrolizumab® (P) as indicated. FIG. 8(A) shows the quantification of co-culture experiment using percentage of dead cells out of total cells (n=3). FIG. 8(B) shows the quantification normalized by cell death observed with drug treatment alone (n=3). Images of a 24-hour co-culture with a 1:1 NK-92 cell to SW480 cell ratio and 5 μM 9-ING-41+25 μg/mL nivolumab® (N) or 25 μg/mL pembrolizumab® (P) as indicated. FIG. 8(C) shows the quantification of co-culture experiment using percentage of dead cells out of total cells (n=3). FIG. 8(D) shows the quantification normalized by cell death observed with drug treatment alone (n=3). EthD-1 was used to visualize dead cells, 10× magnification, scale bar indicates 100 μm. A one-way Anova followed by a post-hoc Dunnett's multiple comparisons test was used to calculate statistical significance. Significance is reported as follows: P≤0.05: *, P≤0.01: **, and P≤0.001: ***.

FIG. 9 shows a regression slope analysis of cytokine profiles shows that 9-ING-41 treatment decreases VEGF and shed PD-L1, while increasing CXCL14 in colorectal cancer cell lines, while 9-ING-41 increases in secreted cytotoxic proteins IFN-gamma, Granzyme B, and TRAIL in immune cells. FIG. 9(A) is a set of eight line graphs showing examples of VEGF, CCL5/RANTES, M-CSF, and PD-L1 secretion into cell culture supernatant with increasing concentrations of 9-ING-41. FIG. 9 also provides heat maps to visualize the regression slopes after forty-eight hour 9-ING-41 treatment of FIG. 9(B) HCT-116, FIG. 9(C) HT-29, and FIG. 9(D) KM12C colorectal cancer cells at IC₁₀, IC₃₀, IC₅₀, and IC₇₀. Red indicates a positive slope while blue indicates a negative slope. FIG. 9 also provides a set of bar graphs showing that FIG. 9(E) TALL-104 cells and FIG. 9(F) NK-92 cells were treated with indicated concentrations of 9-ING-41 for forty-eight hours and cell culture supernatant was analyzed for expression of IFN-gamma, Granzyme B, and TRAIL.

FIG. 10 is a set of eight graphs showing that 9-ING-41 significantly prolongs survival in combination with αPD-L1 therapy in a syngeneic murine colon carcinoma BALB/c murine model using microsatellite stable cell line CT-26. FIG. 10(A) is an experimental model timeline overview for the syngeneic murine colon carcinoma BALB/c murine model using microsatellite stable cell line CT-26. FIG. 10(B) is a graph showing Kaplan-Meier estimator curves for all treatment groups as indicated. Individual Kaplan-Meier estimator curves are shown for FIG. 10(C) 9-ING-41+isotype control, FIG. 10(D) 9-ING-41, FIG. 10(E) anti-PD-1, FIG. 10(F) anti-PD-L1, FIG. 10(G) 9-ING-41+anti-PD-1, and FIG. 10(H) 9-ING-41+anti-PD-L1. Statistical significance was determined using a Log-rank (Mantel-Cox) test.

FIG. 11 is a set of fourteen graphs showing that syngeneic murine colon carcinoma BALB/c murine model with microsatellite stable cell line CT-26 tumor growth curves and mouse body weights grouped by treatment. Individual tumor growth curves for FIG. 11(A) Isotype control, FIG. 11(B) 9-ING-41+isotype control, FIG. 11(C) 9-ING-41, FIG. 11(D) anti-PD-1, FIG. 11(E) anti-PD-L1, FIG. 11(F) 9-ING-41+anti-PD-1, and FIG. 11(G) 9-ING-41+anti-PD-L1. Individual body weight plots for FIG. 11(H) Isotype control, FIG. 11(I) 9-ING-41+isotype control, FIG. 11(J) 9-ING-41, FIG. 11(K) anti-PD-1, FIG. 11(L) anti-PD-L1, FIG. 11(M) 9-ING-41+anti-PD-1, and FIG. 11(N) 9-ING-41+anti-PD-L1.

FIG. 12 is a set of bar graphs showing flow cytometric analyses of cell surface markers of tumor and immune cells treated with 9-ING-41 in vitro. FIG. 12(A) HCT-116, FIG. 12(B) HT-29, FIG. 12(C) TALL-104, and FIG. 12(D) NK-92 cells were treated with indicated concentrations of 9-ING-41 for forty-eight hours, stained for indicated cell surface marker expression, and analyzed with a BD LSRII. Statistical significance was determined using two-tailed unpaired T tests.

FIG. 13 is a drawing showing gating strategies for identification of murine immune cell subsets via multicolor flow cytometry. Cell surface and intracellular markers used for immune cell subtyping via multicolor flow cytometry. Seven-color gating strategy for NK cell panel and for T cell panel.

FIG. 14 is a set of bar graphs showing partial responders have lower percentages of splenic CD4+ T cells and splenic CD8+ T cells, and increased percentages of CD69+ activated T cells, FOXP3+ regulatory T cells, CD3+ TILS, CD4+TILS, and splenic KLRG1+ mature NK cells. 14-days post-treatment initiation, immune cell subpopulations were analyzed in the spleen and tumor. Partial responders (PR) and non-responders (NR) were compared. FIG. 14(A) splenic T cells, FIG. 14(B) tumor-infiltrating T cells, FIG. 14(C) splenic NK cells, and FIG. 14(D) tumor-infiltrating NK cells were compared. Statistical significance was determined using two-tailed unpaired T tests.

FIG. 15 is a set of bar graphs and pie charts showing splenic, but not intra-tumoral, T cell ratios differ between non-responders and partial responders via flow cytometric immunophenotyping analysis. 14-days post-treatment initiation, immune cell subpopulations were analyzed in the spleen and tumor. Partial responders (PR) and non-responders (NR) were compared. T cell ratios were compared in the FIG. 15(A) spleen and FIG. 15(B) tumor. CD11_{high} and CD11_{low} NK cell populations were compared in the FIG. 15(C) spleen and FIG. 15(D) tumor. NK cell subsets based on expression of CD11b and CD27 were compared in the FIG. 15(E-F) spleen and FIG. 15(G-H) tumor. Statistical significance was determined using two-tailed unpaired T tests.

FIG. 16 shows a set of four tables showing a hematologic analysis of murine whole blood samples. Whole blood from long-term mice sacrificed was submitted for serum chemistry analysis. Results from the FIG. 16(A) complete metabolic panel and the FIG. 16(B) complete blood count with differential. See also FIG. 16(C-D), showing codes and descriptions.

FIG. 17 is a pair of tables and line graphs showing twenty-four hour and seventy-two hour 9-ING-41 IC₅₀ calculations and CellTiter-Glo® analysis. The cells were treated as indicted for FIG. 17(A) twenty-four hours or FIG. 17(B) seventy-two hours and then cell viability was assessed.

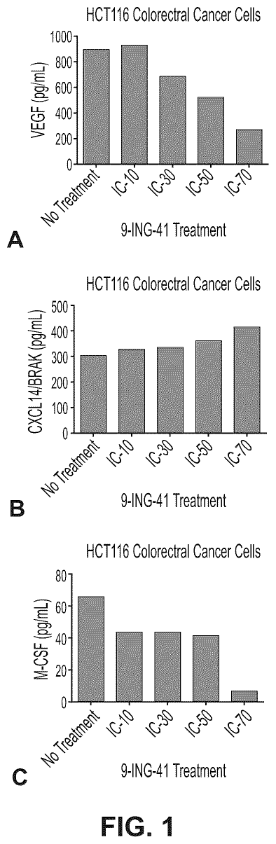

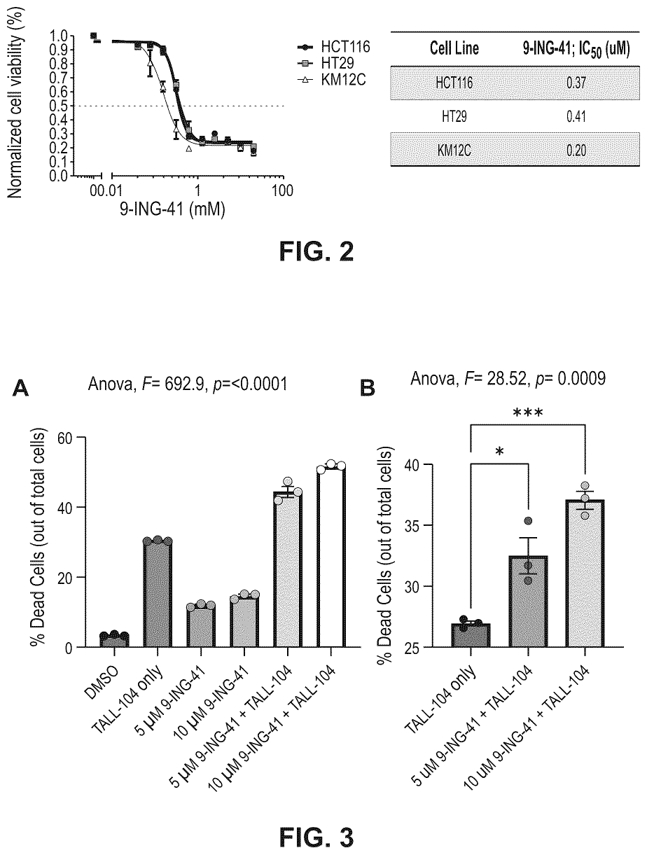

### DETAILED DESCRIPTION OF THE INVENTION

### Industrial Applicability

This invention demonstrates that small-molecule inhibition of GSK-3 can be a potential means to increase efficacy of immune checkpoint blockade and improve response in patients with microsatellite stable colorectal cancer and is predicted to be efficacious in other tumor types. These results support clinical development of 9-ING-41 in combination with αPD-L1 therapy.

Also, evaluating the combination of immune checkpoint blockade with small molecules in oncology represents one of the ways to improve the efficacy of immune checkpoint blockade in microsatellite stable colorectal cancer patients.

### Strategy and Results

The inventors investigated 9-ING-41, a small-molecule that targets GSK-3 which has the potential to increase efficacy of immune checkpoint blockade. GSK-3 inhibitor 9-ING-41 inhibits both α and β isoforms. GSK-3 inhibitor 9-ING-41 is the first clinically relevant small-molecule with superior pharmacokinetic properties that is significantly more potent than other GSK-3 inhibitors. Middha et al., JCO Precis Oncol., 3 (2019). The compound is a reversible ATP-competitive inhibitor that crosses the blood brain barrier and has significant pre-clinical antitumor activity in a broad spectrum of malignancies. Karmali et al., Oncotarget, Vol. 8, No. 70 (2017).

Small-molecule inhibition of GSK-3 using 9-ING-41 leads to increased natural killer and T cell-mediated colorectal cancer cell killing in a co-culture model. 9-ING-41 appears to be acting on tumor cells to sensitize them to immune cell-mediated killing and treatment can boost efficacy of αPD-1 therapy in this in vitro model. This tumor cell sensitization could be because of drug-induced modifications in the tumor cell cytokinome such as decreased VEGF expression, decreased shed PD-L1, and increased CXCL14, as the inventors previously described in Huntington, Louie, Zhou, & EI-Deiry, Oncotarget, Vol. 12, No. 20 (2021). VEGF inhibits T cell activation. Gavalas et al., British Journal of Cancer, 107, 1869-1875 (2012). CXCL14 is a known natural killer cell chemoattractant. Starnes et al., Experimental Hematology, 34, 1101-1105 (2006). Interestingly, the soluble or shed version of PD-L1 can retain the ability to bind PD-1 and function as a decoy receptor to negatively regulate T cell function, despite being a truncated version lacking the membrane domain of the protein. Hassounah et al., Cancer Immunol. Immunother., 68, 407-420 (2019). The increase in efficacy in combination with immune checkpoint blockade in the co-culture model could be due to a concomitant downregulation of shed PD-L1 and an upregulation of cell surface-expressed PD-L1. Another possible mechanism behind 9-ING-41-mediated immunomodulation could be because of a suppression of inflammatory NF-κB signaling or survival pathways in the tumor cells. Treating colorectal cancer cells with 9-ING-41 decreased Survivin, NF-κB p65, and IKBα expression, while increasing PD-L1 expression. This result is consistent with previous studies that showed that GSK-3 is a positive regulator of NF-κB. Medunjanin et al., Sci. Rep. 6, 38553 (2016). Meanwhile, the opposite effect occurred in immune cells, where drug treatment increased NF-κB-inducing kinase (NIK) expression. NIK is the upstream kinase that regulates activation of the non-canonical NF-κB signaling pathway and may implicate a role for non-canonical NF-κB signaling in immune cells post-treatment with 9-ING-41.

The inventors observed differential expression of cell surface markers of tumor and immune cells treated with 9-ING-41 in vitro. GSK-3 inhibition negatively regulates the transcription of PD-1 and LAG-3. Taylor et al., Immunity, 44, 274-286 (2016). The inventors observed a downregulation of PD-1 post-9-ING-41 treatment via Western blot. The inventors conversely observed an upregulation of cell surface LAG-3 expression in immune cells via flow cytometric analysis. GSK-3 induces proteasomal degradation of PD-L1 in tumor cells. Taylor & Rudd, BMC Research Notes, 13, 163-163 (2020). Consistent with these results, the inventors observed an increase in PD-L1 expression post-treatment with 9-ING-41 in tumor cells via Western blot and flow cytometric analysis.

In a syngeneic murine colon carcinoma BALB/c model using MSS cell line CT-26, the authors compared isotype, 9-ING-41, aPD-1, aPD-L1, 9-ING-41+aPD-1, and 9-ING-41+aPD-L1 treatment groups. Monotherapy treatment with 9-ING-41, anti-PD-1 or anti-PD-L1 prolonged the survival compared to the control group. However, they saw the highest probability of survival by Kaplan Meier curve in the 9-ING-41 and anti-PD-L1 combination therapy group, with a 50% probability of survival at 60 days post-treatment initiation (33.3% response rate), compared to 17% for the αPD-1 group (16.6% response rate), and 0% for all other treatment groups (0% response rate). Partial responders had lower percentages of splenic CD4+ T cells and splenic CD8+ T cells and had increased percentages of CD69+activated T cells and FOXP3+ regulatory T cells. The increased splenic percentages of both activated and end-stage T cells in the responder groups could be indicative of an anti-tumor immune response that was mounted earlier in the treatment course. Compared to non-responders, partial responders also had more CD3+ and CD4+ tumor-infiltrating lymphocytes. Further studies will evaluate the contribution of CD4+ versus CD8+ tumor-infiltrating T cells to the observed response to 9-ING-41 and αPD-L1 therapy, especially considering the recent interest in the contribution of helper T cells (CD4+) to anti-tumor immunity. The inventors did not observe many significant differences in splenic NK cell subpopulations in either the tumor or the spleen.

In summary, the drugs were tested in immunocompetent models.

Complete and partial responders have lower serum concentrations of tumorigenic cytokines B-cell activating factor (BAFF), Chemokine ligand 7 (CCL7), CCL12, VEGF, VEGFR2, and CCL21. BAFF is a cytokine that belongs to the TNF ligand superfamily, that may promote tumorigenesis indirectly by induction of inflammation in the tumor microenvironment and directly by induction of epithelial-mesenchymal transition (EMT). Rihacek et al., BioMed Research International 2015, pages 792187-792187. Meanwhile, CCL7 enhances both cancer progression and metastasis via epithelial-mesenchymal transition, including in colon cancer cells. Lee et al., Oncotarget, 7, 36842-36853 (2016). Others have demonstrated that CXCR4 plays a critical role in the promotion of progression of inflammatory colorectal cancer. Yu et al., Journal of Experimental & Clinical Cancer Research, 38, 32 (2019). Expression of VEGF-1 in colorectal cancer is known to be associated with disease localization, stage, and long-term survival. Bendardaf et al., Anticancer Res. 28, 3865-3870 (2008). The inventors previously observed suppression of VEGF in a panel of colorectal cancer lines post-9-ING-41 treatment. Here, a similar suppression of VEGF occurred in the partial and complete responders. The inventors noted a decrease in VEGFR2 in the mice that responded to treatment to some degree. VEGFR2 is known to be highly expressed in colorectal cancer and promotes angiogenesis. Zhong et al., Int. J. Biol. Sci., 16, 272-283 (2020). CCL21, which was also decreased in responder groups, functions in colon cancer metastasis. Li, Sun, Tao, & Wang, Dig. Liver Dis. 43, 40-47 (2011). Many of the analytes that were downregulated in responder groups as compared to non-responder groups function in epithelial-mesenchymal transition.

Complete and partial responders have lower serum concentrations of BAFF, CCL7, CCL12, VEGF, VEGFR2, and CCL21 and higher serum concentrations of CCL4, TWEAK, GM-CSF, CCL22, and IL-12p70 compared to non-responders. Serum from long-term mice sacrificed was analyzed via cytokine profiling for BAFF, CCL7, CCL12, VEGF, VEGFR2, CCL21, CCL4, TWEAK, GM-CSF, CCL22, and (K) IL-12p70. Complete responders, partial responders, and non-responders were compared.

Complete and partial responders had higher serum concentrations of immunomodulatory cytokines of CCL4, TWEAK, GM-CSF, CCL22, and IL-12p70 compared to non-responders. Others demonstrated that CCL4 is an important chemokine in the TME in determining response to immune checkpoint blockade. A lack of CCL4 can lead to the absence of CD103+ dendritic cells (DCs). Williford et al., Science advances, 5, eaayl357-eaayl357 (2019). Dendritic cells are an important cell population influencing the response to immune checkpoint blockade. Thus, they may function in influencing response to therapy.

TWEAK (TNF-related weak inducer of apoptosis) is commonly expressed by peripheral blood monocytes and upregulate its expression after exposure to IFN-γ. Nakayama et al., J. Exp. Med. 192, 1373-1380 (2000). TWEAK promotes the nuclear translocation of both classical and alternative NF-κB pathway subunits. Saitoh et al., TWEAK induces NF-κB2 p100 processing and long-lasting NF-κB activation. J. Biol. Chem., 278, 36005-36012 (2003). Granulocyte-macrophage colony-stimulating factor (GM-CSF) is a well-known immunomodulatory factor that has immunostimulatory functions but it also predictive of poor prognosis in colorectal cancer. See Taghipour et al., Int. J. Mol. Cell Med., 3, 27-34 (2014). CCL22 functions in the recruitment of Tregs to the TME. The high levels of CCL22 in the responders may explain the increased percentage of FOXP3 regulatory T cells observed via flow cytometry within the spleen.

The inventors observed increased levels of IL-12p70 in the responder groups, compared to non-responders. This increase was most pronounced in the 9-ING-41 and anti-PD-L1 treatment group. IL-12 is a potent, pro-inflammatory cytokine that increases activation and cytotoxicity of both natural killer cells and T-cells as well as inhibit immunosuppressive cells, such as tumor associated macrophages (TAMs) and myeloid-derived suppressor cells (MDSCs). See, Watkins, Egilmez, Suttles, & Stout, J. Immunol., 178, 1357-1362 (2007) and Steding et al., Immunology, 133, 221-238 (2011).

GSK-3 inhibitors such as 9-ING-41 represent a possible combination strategy to increase efficacy of immune checkpoint blockade in patients with microsatellite stable colorectal cancer. The 9-ING-41-mediated increase in tumor surface cell-expressed PD-L1 makes this an ideal small molecule to combine with αPD-L1 therapies. The observed decrease in tumor cell survival proteins and increase in immune cell secreted cytotoxic molecules may contribute to the improved survival seen in the 9-ING-41+αPD-L1 treatment group.

### Definitions

For convenience, the meaning of some terms and phrases used in the specification, examples, and appended claims, are listed below. Unless stated otherwise or implicit from context, these terms and phrases shall have the meanings below. These definitions aid in describing particular embodiments but are not intended to limit the claimed invention. Unless otherwise defined, all technical and scientific terms have the same meaning as commonly understood by a person having ordinary skill in the art to which this invention belongs. A term's meaning provided in this specification shall prevail if any apparent discrepancy arises between the meaning of a definition provided in this specification and the term's use in the biomedical art.

9-ING-41 (CAS No. 1034895-42-5; PubChem number; CID 150974278; IU PAC Name: 3-(5-fluoro-1-benzofuran-3-yl)-4-(5-methyl-[1,3]dioxolo[4,5-f]indo1-7-Apyrrole-2,5-dione; elraglusib; chemical structure shown below) is a maleimide-based ATP-competitive and selective inhibitor of glycogen synthase kinase-3 (GSK-3) that stimulates Natural Killer cells and T cells, reduces levels of vascular endothelial growth factor as well as other cytokine, chemokine, and growth factors in colorectal cancer cell supernatant, and stimulates killing of colon cancer cells. 9-ING-41 is with antitumor activity. 9-ING-41 induces apoptosis and cell cycle arrest at prophase by targeting centrosomes and microtubule-bound GSK3β. 9-ING-41 is commercially available from Selleck Chemicals, Houston, TX, USA (Catalog No. S9602). See Ugolkov et al., Anticancer Drugs, 29(8), 717-724 (September 2018).

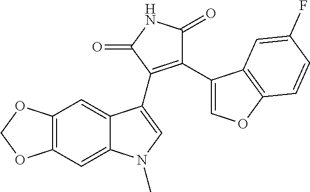 [SMILES: Cn1cc(C2=C(c3coc4ccc(F)cc34)C(=O)NC2=O)c2cc3c(cc21)OCO3]

A or an means at least one or one or more unless the context indicates otherwise.

About means that the recited numerical value is approximate. Small variations would not significantly affect the practice of the disclosed embodiments. Where a numerical value is used unless indicated otherwise by the context, about means the numerical value can vary by ±10% and remain within the scope of the disclosed embodiments.

Acylamino has the organic chemical art-recognized meaning of an amino group substituted by an acyl group.

Alkenyl has the organic chemical art-recognized meaning of a straight or branched alkyl group having 2 to 20 carbon atoms and having one or more double carbon-carbon bonds.

Alkoxy has the organic chemical art-recognized meaning of a straight or branched —O-alkyl group having 1 to 20 carbon atoms.

Alkyl has the organic chemical art-recognized meaning of a saturated hydrocarbon group which is straight-chained or branched.

Alkylamino has the organic chemical art-recognized meaning of an amino group substituted by an alkyl group. In some embodiments, the alkyl group is a lower alkyl group having from 1 to 6 carbon atoms.

Alkylene or alkylenyl has the organic chemical art-recognized meaning of a divalent alkyl linking group.

Alkylthio has the organic chemical art-recognized meaning of an —S-alkyl group having from 1 to 6 carbon atoms.

Alkynyl has the organic chemical art-recognized meaning of a straight or branched alkyl group having 2 to 20 carbon atoms and one or more triple carbon-carbon bonds.

Amidino has the organic chemical art-recognized meaning of —C(═NH)NH₂.

Amino has the organic chemical art-recognized meaning of —NH 2.

Aminoalkoxy has the organic chemical art-recognized meaning of an alkoxy group substituted by an amino group.

Aminoalkyl has the organic chemical art-recognized meaning of an alkyl group substituted by an amino group.

Animal includes, but is not limited to, mammals, humans, and non-human vertebrates, such as wild, domestic, and farm animals.

Antagonize, and antagonizing has the organic chemical art-recognized meaning of reducing or eliminating one or more effects.

Approximately or About means that a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). Reference to approximately or about a value or parameter includes (and describes) embodiments directed to that value or parameter. For example, a description referring to about X includes a description of X.

Aryl has the organic chemical art-recognized meaning of a monocyclic, bicyclic, or polycyclic aromatic hydrocarbon.

Arylalkyl has the organic chemical art-recognized meaning of an alkyl group substituted by an aryl. In some embodiments, an alkyl group is a C₁₋₆ alkyl group.

Arylamino has the organic chemical art-recognized meaning of an amino group substituted by an aryl group.

Arylene has the organic chemical art-recognized meaning of an aryl linking group, i.e., an aryl group that links one group to another in a molecule.

Beneficial or desired clinical results include, but are not limited to, alleviation of one or more symptom(s), diminishment of the extent of the deficit, stabilized (i.e., not worsening) state of a tumor or malignancy, delay or slowing of tumor growth or metastasis, and an increased lifespan as compared to that expected in the absence of treatment.

Benign or non-malignant means tumors that may grow larger but do not spread to other parts of the body. Benign tumors are self-limited and typically do not invade or metastasize.

C₁₋₆ alkyl is specifically intended to individually disclose methyl, ethyl, propyl, C₄alkyl, C₅alkyl, and C₆alkyl.

Cancer Cell or Tumor Cell means an individual cell of a cancerous growth or tissue. A cancer cell is a cancerous, pre-cancerous, or transformed cell, either in vivo, ex vivo, or in tissue culture, with spontaneous or induced phenotypic changes that do not necessarily involve the uptake of new genetic material. Although transformation can arise from infection with a transforming virus and incorporation of new genomic nucleic acid or uptake of exogenous nucleic acid, it can also occur spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor-specific markers, invasiveness or metastasis, and tumor growth in suitable animal hosts such as nude mice.

Cancer therapy has the medical art-recognized meaning of anticancer treatment to cure or prolong the life of a mammal with cancer, especially a human with cancer. Among the cancer therapies known in the medical art include the following: Some therapies treat tumors with mutated p53. Some chemotherapies involve administering 5-fluorouracil (5-FU), irinotecan, etoposide, gemcitabine, oxaliplatin, carboplatin, paclitaxel, or a combination thereof to the subject who has cancer. Some radiotherapies involve administering radiation to the subject who has cancer. Some chemotherapies involve administering PARP inhibitors. Some therapies target. DNA repair-deficient cancers that may have defective repair of replicating DNA. Examples of DNA repair-deficient cancers include BRCA1-deficient cancers. Some chemotherapies involve administering immune checkpoint therapy, such as anti-PD-1, anti-PD-L1, or anti-CTLA4 antibodies. Some chemotherapies are targeted cancer therapies that involve administering anti-ATM, anti-ATR, anti-Chk1, anti-Chk2, anti-EGFR, anti-alk, anti-Her2, anti-NTRK, anti-BRAF, anti-KRAS antibodies to the subject who has cancer.

Carrier has the medical chemical art-recognized meaning of a diluent, adjuvant, or excipient with which a compound is administered in a composition.

Compound has the medical chemical art-recognized meaning of all stereoisomers, tautomers, isotopes, and polymorphs of the compounds.

Comprising (and any form of comprising, such as comprise, comprises, and comprised), having (and any form of having, such as have and has), including (and any form of including, such as includes and include), or containing (and any form of containing, such as contains and contain), are inclusive and open-ended and include the options following the terms, and do not exclude additional, unrecited elements or method steps.

Contacting has the medical chemical art-recognized meaning of bringing together two compounds, molecules, or entities in an in vitro system or an in vivo system.

COVID-19 has medical chemical art-recognized meaning of an infectious disease caused by the SARS-CoV-2 virus.

Cyano has the organic chemical art-recognized meaning of —CN.

Cycloalkyl has the organic chemical art-recognized meaning of non-aromatic cyclic hydrocarbons, including cyclized alkyl, alkenyl, and alkynyl groups that have up to 20 ring-forming carbon atoms.

Darolutamide (DARO) is an androgen receptor (AR) antagonist approved for the treatment of patients with non-metastatic CRPC. DARO has higher affinity to AR and preclinical activity against enzalutamide-resistant PC cell lines, including AR variants associated with enzalutamide agonism. Borgmann, Eur. Urol. (2018).

DR5 has the molecular biological art-recognized meaning of protein on the surface of some cells that binds another protein called TRAIL, which may kill some cancer cells. An increase in the amount or activity of DR5 on cancer cells may kill more cells. Also called death receptor 5, TRAIL receptor 2, TRAIL-R2, and tumor necrosis factor receptor superfamily member 10B. See National Cancer Institute (NCI) Dictionary of Cancer Terms.

Effective Amount and Therapeutically Effective Amount include an amount sufficient to prevent or ameliorate a manifestation of the disease or medical condition, such as colorectal cancer. Many ways are known in the biomedical art to determine the effective amount for a given application. For example, pharmacological methods for dosage determination can be used in the therapeutic context. In the context of therapeutic or prophylactic applications, the amount of a composition administered to the subject depends on the type and severity of the disease and on the characteristics of the individual, such as general health, age, sex, body weight, and tolerance to drugs, and on the degree, severity, and type of disease. The skilled artisan can determine appropriate dosages depending on these and other factors. The compositions can also be administered in combination with one or more additional therapeutic compounds.

ERK1/2 is a protein in the extracellular signal-regulated kinase 1/2 (ERK1/2) cascade, a central signaling pathway that regulates a wide variety of stimulated cellular processes, including mainly proliferation, differentiation, and survival, but also apoptosis and stress response.

Glycogen synthase kindase-3 (GSK-3) is a ubiquitously expressed protein kinase that exists in two isoforms (α, β), and is constitutively active. GSK-3 promotes the growth of some cancers, such as pancreatic cancers and colorectal cancers. See Ding et al., Clin. Cancer Res., 25, 6452-6462 (2019); Li J et al., Gastroenterology, 128, 1907-1918 (2005). GSK-3 is a positive regulator of NF-κB. GSK-3 promotes cancer cell survival and proliferation by facilitating chemoresistance. Medunjanin et al., Sci. Rep. 6, 38553 (2016). GSK-3 functions both as a proto-oncogene and as a tumor suppressor. Keith et al., International Journal of Cell Biology (2012). GSK-3 can phosphorylate β-catenin, triggering β-catenin destabilization and degradation, and maintaining low levels of β-catenin in the cytosol and nucleus. Inhibition of GSK-3 can lead to stabilization and activation of β-catenin, which could activate proliferative, tumorigenic pathways. Li J et al., Gastroenterology, 128, 1907-1918 (2005). GSK-3 is necessary for NF-κB signaling through modulation of NEMO phosphorylation. GSK-3 inhibition could suppress this inflammatory pathway. Medunjanin et al., Sci. Rep. 6, 38553 (2016). GSK-3 regulates the expression of checkpoint ligands in both immune and cancer cells. In CD8+ T cells, GSK-3 inhibition regulates PD-1 transcription and enhances T cell function. Taylor et al., Immunity, 44, 274-286 (2016).

Halo has the organic chemical art-recognized meaning of halogen groups.

Haloalkoxy has the organic chemical art-recognized meaning of an —O— haloalkyl group.

Haloalkyl has the organic chemical art-recognized meaning of a C1-6alkyl group having one or more halogen substituents.

Heteroaryl has the organic chemical art-recognized meaning of an aromatic heterocycle having up to 20 ring-forming atoms (e.g., C) and having at least one heteroatom ring member (ring-forming atom) such as sulfur, oxygen, or nitrogen.

Heteroarylalkyl has the organic chemical art-recognized meaning of a C₁₋₆ alkyl group substituted by a heteroaryl group.

Heteroarylamino has the organic chemical art-recognized meaning of an amino group substituted by a heteroaryl group.

Heteroarylene has the organic chemical art-recognized meaning of a heteroaryl linking group, i.e., a heteroaryl group that links one group to another group in a molecule.

Heterocycle or heterocyclic ring has the organic chemical art-recognized meaning of a 5- to 7-membered monocyclic or 7- to 10-membered bicyclic ring system, any ring of which may be saturated or unsaturated, which ring consists of carbon atoms and from one to three heteroatoms chosen from N, O and S, wherein the N and S heteroatoms may optionally be oxidized. The N heteroatom may optionally be quaternized, including by any bicyclic group in which any of the above-defined heterocyclic rings is fused to a benzene ring.

Heterocycloalkyl has the organic chemical art-recognized meaning of non-aromatic heterocycles having up to 20 ring-forming atoms, including cyclized alkyl, alkenyl, and alkynyl groups, where one or more of the ring-forming carbon atoms is replaced by a heteroatom, such as an O, N, or S atom. Hetercycloalkyl groups can be monocyclic or polycyclic (e.g., fused, bridged, or spiro systems).

Hydroxy or hydroxyl has the organic chemical art-recognized meaning of an —OH group.

Hydroxyalkyl or hydroxylalkyl has the organic chemical art-recognized meaning of an alkyl group substituted by a hydroxyl group.

In need thereof has the medical chemical art-recognized meaning of that the individual, subject, or patient has been identified as needing the method, prevention, or treatment. The identification can be by any diagnostic method. In any of the methods, preventions, and treatments described herein, the individual, subject, or patient can be in need thereof. The individual, subject, or patient may be in an environment or will be traveling to an environment or has traveled to an environment in which a disease, disorder, or condition is prevalent.

Individual, subject, and patient, used interchangeably, mean any animal. In some embodiments, the mammal is a human.

Integer means a numerical value that is a whole number. For example, an integer from 1 to 5 means 1, 2, 3, 4, or 5.

Isolated has the medical chemical art-recognized meaning of that the compounds, or pharmaceutically acceptable salts thereof, are separated from other components of either (a) a natural source, such as a plant or cell, such as bacterial culture, or (b) a synthetic organic chemical reaction mixture, such as by conventional techniques.

Mammal has the medical chemical art-recognized meaning, and includes rodents, monkeys, and humans. In some embodiments, the mammal is a human.

N-membered, where n is an integer, typically describes the number of ring-forming atoms in a moiety, where the number of ring-forming atoms is n. For example, pyridine is an example of a 6-membered heteroaryl ring, and thiophene is an example of a 5-membered heteroaryl ring.

Nitro has the organic chemical art-recognized meaning of —NO 2.

Optionally substituted has the organic chemical art-recognized meaning of that a substitution is optional and, therefore, includes both unsubstituted and substituted atoms and moieties. A substituted atom or moiety indicates that any hydrogen atom on the designated compound or moiety can be replaced with a selection from the mentioned substituent groups, provided that the normal valency of the designated compound or moiety is not exceeded, and that the substitution results in a stable compound.

p53 pathway restoration has the cell-biological art-recognized meaning of a medical intervention effort to restore p53 activity as an anticancer therapeutic approach. See Martinez, Restoring p53 tumor suppressor activity as an anticancer therapeutic strategy. Future Oncol. 6(12), 1857-1862 (December 2010). The S-phase DNA damage response pathway is characterized by the increase in p-ATR (Thr1989). This increase ultimately leads to a delay in S-phase cells. This S-phase perturbation may contribute to cancer cell death.

p53, a tumor-suppressor, prevents cancer development via initiating cell-cycle arrest, cell death, repair, or anti-angiogenesis processes. Over 50% of human cancers harbor cancer-causing mutations in p53. p53 mutations not only abrogate its tumor-suppressor function, but also endow mutant p53 with a gain of function (GOF), creating a proto-oncogene that contributes to tumorigenesis, tumor progression, and chemotherapy or radiotherapy resistance. Targeting mutant p53 or restoring a wild-type p53 signaling pathway provides an attractive strategy for cancer therapy.

Pharmaceutically acceptable has the medical chemical art-recognized meaning that the compounds, materials, compositions, or dosage forms are within the scope of sound medical judgment and are suitable for contact with tissues of humans and other animals. The pharmaceutically acceptable compounds, materials, compositions, or dosage forms result in no persistent detrimental effect on the subject or the general health of the treated subject. Still, transient effects, such as minor irritation or a stinging sensation, are common with the administration of medicament and are consistent with the composition, formulation, or ingredient (e.g., excipient) in question. Guidance as to what is pharmaceutically acceptable is provided by comparable compounds, materials, compositions, or dosage forms listed in the US Pharmacopeia or another generally recognized pharmacopeia for use in animals, and more particularly in humans.

Pharmaceutically acceptable salts include, but are not limited to, salts of acidic or basic groups. Basic compounds can form a wide variety of salts with various inorganic and organic acids. Compounds that include an amino moiety may form pharmaceutically acceptable salts with various amino acids. Acidic compounds can form base salts with different pharmacologically acceptable cations. Salts include quaternary ammonium salts of the compounds described herein, where the compounds have one or more tertiary amine moiety.

Phenyl has the organic chemical art-recognized meaning of —C₆H₅. A phenyl group can be unsubstituted or substituted with one, two, or three suitable substituents.

Prevention or preventing has the medical chemical art-recognized meaning of reducing the risk of acquiring a disease, condition, or disorder.

Prodrug has the medical chemical art-recognized meaning of a derivative of a known direct-acting drug, which derivative may have enhanced delivery characteristics and therapeutic value as compared to the active drug and is transformed into the active drug by an enzymatic or chemical process. Prodrugs can be prepared by modifying functional groups present in the compounds so that the modifications are cleaved, either in routine manipulation or in vivo, to the parent compounds.

Purified has the medical chemical art-recognized meaning of that when isolated, the isolate contains at least 90%, at least 95%, at least 98%, at least 99%, or 100% of a compound by weight of the isolate.

Quaternary ammonium salts have the medical chemical art-recognized meaning of derivatives of the disclosed compounds with one or more tertiary amine moieties wherein at least one of the tertiary amine moieties in the parent compound is modified by converting the tertiary amine moiety to a quaternary ammonium cation via alkylation.

S-phase perturbation means disruption or delay of the normal passage of cells through the S-phase of the cell cycle during which DNA replication occurs (where the DNA content of the cell doubles from 1N content (haploid) to 2N content (diploid). This can occur through activation of an S-phase checkpoint which represents a response to cellular sensing of disrupted DNA replication or disrupted cell signaling of processes important for passage of cells through the S-phase of the cell cycle (during which DNA replication occurs.

Solubilizing agent has the medical chemical art-recognized meaning of agents that result in the formation of a micellar solution or a true solution.

Solution/suspension has the medical chemical art-recognized meaning of a liquid composition wherein a first portion of the active agent is present in solution. A second portion of the active agent is present in particulate form, in suspension in a liquid matrix.

Subject That Has A Cancer or Subject That Has A Tumor is a subject having objectively measurable cancer cells present in the subject's body. Included in this definition are malignant, actively proliferative cancers and potentially dormant tumors or micrometastases. Cancers that migrate from their original location and seed other vital organs can eventually lead to the death of the subject through the functional deterioration of the affected organs. Hemopoietic cancers, such as leukemia, can out-compete the regular hemopoietic compartments in a subject, thereby leading to a hemopoietic failure (in the form of anemia, thrombocytopenia, and neutropenia), ultimately causing death. A subject can have been previously diagnosed with or identified as suffering from or having a condition in need of treatment (e.g., a cancer) or one or more complications related to such a condition, and optionally, but need not have already undergone treatment for a condition or the one or more complications related to the condition. A subject can also not have been previously diagnosed as having a condition in need of treatment or one or more complications related to such a condition. For example, a subject can exhibit one or more risk factors for a condition, or one or more complications related to a condition or a subject who does not exhibit risk factors.

Substantially isolated has the medical chemical art-recognized meaning of a compound that is at least partially or substantially separated from the environment in which it is formed or detected.

Suitable substituent or substituent has the medical chemical art-recognized meaning of a group that does not nullify the synthetic or pharmaceutical utility of the compounds or the intermediates useful for preparing them. One of skill in medical chemical art can readily choose a suitable substituent based on the stability and pharmacological and synthetic activity of the compounds described herein.

Therapeutically effective amount has the medical chemical art-recognized meaning of the amount of active compound or pharmaceutical agent that elicits the biological or medicinal response that is being sought in a tissue, system, animal, individual or human by a researcher, veterinarian, medical doctor, or another clinician. The therapeutic effect is dependent upon the disorder being treated or the biological effect desired. As such, the therapeutic effect can be a decrease in the severity of symptoms associated with the disorder or inhibition (partial or complete) of progression of the disorder, or improved treatment, healing, prevention or elimination of a disorder, or side-effects. The amount needed to elicit the therapeutic response can be based on, for example, the age, health, size, and sex of the subject. Optimal amounts can also be determined based on monitoring of the subject's response to treatment.

Treat, treated, or treating has the medical chemical art-recognized meaning of both treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of the condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of the condition, disorder or disease. Treatment includes eliciting a clinically significant response, optionally without excessive levels of side effects. Treatment also includes prolonging survival as compared to expected survival if not receiving treatment.

Treat, Treatment, Treating, or Amelioration Of A Disease, Disorder Or Medical Condition means therapeutic treatments for a condition. The object is to reverse, alleviate, ameliorate, inhibit, slow down or stop the progression or severity of a symptom or condition. The term treating includes reducing or alleviating at least one adverse effect or symptom of a condition. Treatment is generally effective if one or more symptoms or clinical markers are reduced. Alternatively, treatment is effective if the progression of a condition is reduced or halted. That is, treatment includes not just the improvement of symptoms or markers but also a cessation or at least slowing of progress or worsening of symptoms that would be expected in the absence of treatment.

Unless otherwise defined herein, scientific and technical terms used with this application shall have the meanings commonly understood by persons having ordinary skill in the biomedical art. This invention is not limited to the particular methodology, protocols, and reagents, etc., described herein and as such can vary.

The disclosure described herein does not concern a process for cloning humans, processes for modifying the germ line genetic identity of humans, uses of human embryos for industrial or commercial purposes or processes for modifying the genetic identity of animals which are likely to cause them suffering with no substantial medical benefit to man or animal, and also animals resulting from such processes.

Guidance from Materials and Methods

A person having ordinary skill in the art can use these materials and methods as guidance to predictable results when making and using the invention:

Cell culture maintenance. Human colorectal cancer cells SW480, HCT-116, HT-29, and KM12C were used in this study. SW480 cells were cultured in Dulbecco's Modified Eagle Medium (DMEM) supplemented with 10% FBS and 1% Penicillin-Streptomycin HCT-116 and HT-29 were cultured in McCoy's 5A (modified) Medium supplemented with 10% FBS and 1% Penicillin-Streptomycin. KM12C cells were cultured in Eagle's Minimal Essential Medium supplemented with 10% FBS and 1% Penicillin-Streptomycin. Human immune cells NK-92, TALL-104, and patient-derived CD8+ T cells were also used in this study. NK-92 cells were cultured in Alpha Minimum Essential medium supplemented with 2 mM L-glutamine, 1.5 g/L sodium bicarbonate, 0.2 mM inositol, 0.1 mM 2-mercaptoethanol, 0.02 mM folic acid, 12.5% horse serum, and 12.5% FBS. TALL-104 cells (CD2+; CD3+; CD7+; CD8+; CD56+; CD4−; CD16−) and patient-derived T cells (CD3+; CD8+) were cultured in RPMI-1640 containing 20% FBS, 100 Wml penicillin, and 100 μg/ml streptomycin. Recombinant human IL-2 (Miltenyi cat #130-097744) with a final concentration of 100 units/mL was added all immune cell culture media. All cell lines were incubated at 37° C. in a humidified atmosphere containing 5% CO₂. Cell lines were authenticated and tested to ensure the cultures were free of mycoplasma infection.

Measurement of cell viability. Cells were seeded at a density of 3×10³ cells per well in a 96-well plate (Greiner Bio-One, Monroe, NC, USA). Cell viability was assessed using the CellTiter Glo assay (Promega, Madison, WI, USA). Cells were mixed with 25 μL of CellTiter-Glo reagents in 100 μL of culture volume, and bioluminescence imaging was measured using the Xenogen IVIS imager (Caliper Life Sciences, Waltham, MA, USA). The percent of cell viability was determined by normalizing luminescence signal to control wells. Dose-response curves were generated, and the half maximal inhibitory concentration (1C-50) was calculated using Graph-Pad Prism version 9.2.0.

Isolation of donor derived CD8+ T cells. An Easy Step Human CD8+ T Cell Isolation Kit was used to isolated CD8+ T cells from a donor PBMC sample via negative selection (Cat #, 17913, Stem Cell Technologies, Vancouver, Canada).

Collection of cell culture supernatants used in cytokine measurements. Cells were plated at 3.5×10⁴ cells in a 48-well plate (Thermo Fisher Scientific, Waltham, MA, USA) in complete medium and incubated at 37° C. with 5% CO₂. At twenty-four hours after plating, almost all the tumor cells were adherent to the bottom of the flask and the complete medium was replaced with drug-containing medium. Subsequently, the culture supernatants were collected after forty-eight hours of incubation and were frozen at −80° C. until the measurement of cytokines was performed. The day of analysis, samples were thawed and centrifuged to remove cellular debris.

Human cytokine profiling. Human cell line culture supernatants were analyzed using an R&D systems Human Premixed Multi-Analyte Kit (R&D Systems, Inc., Minneapolis, MN, USA) and a Luminex 200 Instrument (LX200-XPON-RUO, Luminex Corporation, Austin, TX, USA) according to the manufacturer's instructions. Sample levels of TNF-α, 4-1BB/TNFRSF9/CD137, IL-8/CXCL8, Ferritin, IFN-β, IL-10, CCL2/JE/MCP-1, VEGF, CXCL13/BLC/BCA-1, IFN-γ, CCL20/MIP-3 α, CCL3/MIP-1 α, CCL22/MDC, CCL4/MIP-1 β, Fas Ligand/TNFSF6, IL-17/IL-17A, IL-2, BAFF/BLyS/TNFSF13B, GM-CSF, CXCLS/ENA-78, TRANCE/TNFSF11/RANK L, CXCL9/MIG, G-CSF, IFN-γ R1/CD119, VEGFR3/Flt-4, C-Reactive Protein/CRP, CXCL11/I-TAC, IL-21, CXCL14/BRAK, IL-6, Fas/TNFRSF6/CD95, TRAIL R3/TNFRSF10C, IL-4, CCL5/RANTES, PD-L1/B7-H1, CCL7/MCP-3/MARC, Chitinase 3-like 1, CXCL10/IP-10/CRG-2, IL-1β/IL-1F2, IL-7, Prolactin, CCL8/MCP-2, TRAIL R2/TNFRSF10B, M-CSF, IL-15, Granzyme B, IFN-α, TREM-1, IL-12/IL-23 p40, TRAIL/TNFSF10, CCL11/Eotaxin, and IL-18/IL-1F4. Sample values are reported in picograms per milliliter (pg/mL).

Murine cytokine profiling. Whole blood from mice was collected and allowed to clot. Serum was isolated using a serum separator tube (SST) according to manufacturer instructions. Murine serum samples were analyzed using an R&D systems Murine Premixed Multi-Analyte Kit (R&D Systems, Inc., Minneapolis, MN, USA) and a Luminex 200 Instrument (LX200-XPON-RUO, Luminex Corporation, Austin, TX, USA) according to the manufacturer's instructions. Sample levels of GM-CSF, IL-7, IL-12 p70, CCL2/JE/MCP-1, IL-1 β/IL-1F2, VEGF, IL-2, IL-4, VEGFR2/KDR/Flk-1, IL-6, IL-10, IL-β, IFN-γ, IL-3, IL-16, CXCL10/IP-10/CRG-2, CCL5/RANTES, CCL7/MCP-3/MARC, CCL12/MCP-5, Prolactin, M-CSF, CCL3/MIP-1 α, IL-1 α/IL-1F1, CCL20/MIP-3 α, CCL4/MIP-1 β, TWEAK/TNFSF12, CXCL12/SDF-1 α, BAFF/BLyS/TNFSF1B, Granzyme B, CCL21/6Ckine, CCL11/Eotaxin, and CCL22/MDC. Sample values are reported in picograms per milliliter (pg/mL). A quantitative analysis with 6 standards and a minimum of 50 counts per bead region was used with the Luminex to generate analyte values reported as picograms/milliliter (pg/mL). Cytokine concentrations less than the lower limit of detection for each cytokine were recoded as zero. Cytokines without detectable expression levels were removed from further analysis of each cell line. Cytokine dose-response effect was modeled by simple linear regression for each drug. The slopes of the linear regressions were compared. Data analysis and visualization were generated using R (R Development Core Team, 2020).

Immune cell co-culture experiments. Co-culture experiments were conducted with GFP+ SW480 or HCT-116 colorectal cancer cells and either NK-92 natural killer cells or TALL-104 T cells in a 48-well plate, in the presence or absence of 9-ING-41. HCT-116 cells were labeled using CellTracker™ Green CMFDA (5-chloromethylfluorescein diacetate), immune cells (NK-92, TALL-104) were labeled using CellTracker™ Blue CMAC Dye (7-amino-4-chloromethylcoumarin), and ethidium homodimer-1 (EthD-1) was used as a marker of cell death (Invitrogen, Thermo Fisher Scientific, Waltham, MA, USA).

Generation of single cell suspensions. Spleens were strained, filtered, and washed while tumors were collected, washed, and digested before lymphocytes were collected using a Percoll gradient (Cat #P1644-100ML, Sigma Aldrich, St. Louis, MO, USA).

Flow cytometry. Flow cytometry viability staining was conducted by suspending murine spleen and tumor single cell suspensions in Zombie Violet fixable viability kit (Cat #423114, BioLegend, San Diego, CA, USA) according to manufacturer instructions for thirty minutes at room temperature. Staining for membrane surface proteins was conducted using conjugated primary antibodies for one hour on ice, according to manufacturer instructions. Cells were fixed and permeabilized using the eBioscience™ Foxp3/Transcription Factor Staining Buffer Set according to manufacturer instructions (Cat #00-5523-00, Invitrogen, Thermo Fisher Scientific, Waltham, MA, USA). Cells were resuspended in Flow Cytometry Staining Buffer (R&D Systems, Minneapolis, MN, USA) and were analyzed using a BD Biosciences LSR II and FlowJo version 10.1 (FlowJo, Ashland, OR, USA).

Natural killer cell immunophenotyping. The NK cell flow cytometry panel included the following directly conjugated primary antibodies: Anti-mouse CD45, eBioscience eVolve 605 clone: 30-F11 (Ref #83-0451-42, Invitrogen, Thermo Fisher Scientific, Waltham, MA, USA), PE anti-mouse CD3 molecular complex (17A2) (mat. #: 555275, BD Biosciences, Franklin Lakes, NJ, USA), Anti-mouse NKp46 APC (Ref #17-3351-82), APC/Cy7 anti-mouse/human CD11 b clone: M1/70 (cat #101226, BioLegend), anti-Cd27 Monoclonal Antibody (LG.7F9) FITC (eBioscience™, Thermo Fisher Scientific, Waltham, MA, USA, cat #11-0271-82), and (KIrgl Monoclonal Antibody (2F1) PE-Cyanine7 (eBioscience™, Thermo Fisher Scientific, Waltham, MA, USA, cat #25-589382). Gating strategies are as follows:

NK cell: live/CD45/CD3−/NK1.1+

Mature NK cell: live/CD45/CD3−/NK1.1+/KRLG1+

Activated NK cell: live/CD45/CD3−/NK1.1+/CD11b+

NK cell subset 1: live/CD45/CD3−/NK1.1+/CD11b-CD27−

NK cell subset 2: live/CD45/CD3−/NK1.1+/CD11b-CD27+

NK cell subset 3: live/CD45/CD3−/NK1.1+/CD11b+CD27+

NK cell subset 4: live/CD45/CD3−/NK1.1+/CD11b+CD27−

T cell immunophenotypind. The T cell flow cytometry panel included the following directly-conjugated primary antibodies: Anti-mouse CD45 superbright 600 clone: 30-511 (ref #63-0451-82, eBioscience), anti-CD3 APC-Cy7 clone 17A2(BD Biosciences, cat #560590), eBioscience anti-mouse CD4 PE-Cy7 clone: RM4-5 (Ref #25-0042-82, Invitrogen), PE anti-mouse CD8a (Ly-2)(53-6.7) (cat #553032, BD), Anti-mouse CD69 FITC clone: H1.2F3 (Ref #11-0691-81, eBioscience), and FOXP3 (FJK-16s) APC (eBioscience). Gating strategies are as follows:

CD4+ T cell: live/CD45+/CD3+/CD4+/FOXP3−

CD8+ T cell: live/CD45+/CD3+/CD8+

Treg: live/CD45+/CD3+/CD4+/FOXP3+

Activated CD8+ T cell: live/CD45+/CD3+/CD8+/CD69+

Western blot analysis. Cells were plated in a 6-well plate and incubated overnight before the spent media was replaced with drugged media. Drug treatment lasted for indicated durations. Protein was extracted using radioimmunoprecipitation (RIPA) assay buffer (Cat #R0278, Sigma-Aldrich, St. Louis, MO) containing cOmplete™ Mini, EDTA-free Protease Inhibitor Cocktail (Cat #4693159001, Roche, Basel, Switzerland). Denaturing sample buffer was added, samples were boiled at 95 degrees for 10 minutes. An equal amount of protein lysate was electrophoresed through NuPAGE™ 4 to 12%, Bis-Tris, 1.5 mm, Mini Protein Gels (Invitrogen, Waltham, MA, USA) then transferred to PVDF membranes. The PVDF membrane was blocked with 5% non-fat milk (Sigma-Aldrich, St. Louis, MO, USA) in 1× TTBS. Primary antibodies were incubated with the transferred PVDF membrane in blocking buffer at 4° C. overnight. Secondary antibodies used included goat anti-rabbit IgG (H+L) secondary antibody, HRP (Cat #31460, Invitrogen, Waltham, MA, USA) and goat anti-mouse IgG (H+L) secondary antibody, HRP (Cat #31430, Invitrogen, Waltham, MA, USA). Signal was detected using a Syngene Imaging System.

In vivo studies. The experimental in vivo protocol (Protocol #19-01-003) as approved by the Institutional Animal Care and Use Committee of Brown University (Providence, RI, USA). Six to seven weeks old male BALB/c mice were purchased from Taconic. 50,000 cells were suspended in 50 μL phosphate-buffered serum and 50 μL Matrigel (Thermo Fisher Scientific, catalog no. 354234). 100 μL was injected subcutaneously into the rear flanks. Once tumor volume reached at least 100 mm3, mice were randomly assigned to one of four groups (three mice/group): vehicle, ONC212, 2-DG, combination of ONC212 p2-DG. ONC212 was delivered by oral gavage at the dosage of 50 mg/kg in a solution of 70% phosphate-buffered serum, 10% DMSO, and 20% Kolliphor EL (Sigma-Aldrich, catalog no. C5135), three times per week. The treatment continued until mice developed signs of toxicity or discomfort from excessive tumor growth. Mice were weighed once a week to monitor signs of drug toxicity. The length (L) and width (VV) of the masses were measured three times per week with a digital caliper. The tumor volume was calculated applying the formula: 0.5LW2. Collection of whole blood and serum were performed by cardiac puncture and sent to Antech GLP for blood cell count and chemistry tests. Tumors and organs were dissected and harvested for immunohistochemistry.

Immunohistochemistry. Excised tissues are fixed with 10% neutral buffered formalin and paraffin embedded. 5 micrometer tissue sections are cut with a microtome and mounted on glass microscope slides for staining. Hematoxylin and eosin staining was completed for all tumor specimens. Paraffin embedding and sectioning of slides were performed by the Brown University Molecular Pathology Core Facility. Slides were dewaxed in xylene and subsequently hydrated in ethanol at decreasing concentrations. Antigen retrieval was carried out by boiling the slides in 2.1 g citric acid (pH 6) for 10 minutes. Endogenous peroxidases were quenched by incubating the slides in 3% hydrogen peroxide for 5 minutes. After nuclear membrane permeabilization with Tris-buffered saline plus 0.1% Tween 20, slides were blocked with horse serum (Vector Laboratories, catalog no. MP-7401-15), and incubated with primary antibodies overnight in a humidified chamber at 4° C. After washing with phosphate-buffered serum, secondary antibody (Vector Laboratories, catalog no. MP-7401-15 or MP-7402) was added for thirty minutes, followed by diaminobenzidine application (Thermo Fisher Scientific, Waltham, MA, USA, catalog no. NC9276270), according to the manufacturer's protocol. Samples were counterstained with hematoxylin, rinsed with distilled water, dehydrated in an increasing gradient of ethanol, cleared with xylene, and mounted with Cytoseal mounting medium (Thermo Fisher Scientific, Waltham, MA, USA, catalog no. 8312-4). Images were recorded on Axioskop microscope (Zeiss), using QCapture. QuPath software was used to automatically count positive cells. For each immunohistochemistry marker, five 20×images per group were analyzed. Results were represented as the absolute number of positive cells per 20×field.

Microarrays. A total of 0.5×106 tumor cells (HCT-116, HT-29, KM12C) were plated in a 6 well plate and allowed to adhere overnight before 24-hour treatment as indicated. 1×106 immune cells (NK92, TALL-104) were plated and treated with 9-ING-41 as indicated for twenty-four hours. RNA was isolated from cell pellets in batches of 6 using a RNeasy plus Mini Kit (Cat #74134, Qiagen, Hilden, Germany). Acceptable RNA concentration and quality were verified with Nanodrop and Bioanalyzer measurements. GeneChip™ Human Transcriptome Array 2.0 assays were conducted according to manufacturer instructions in two batches using randomized samples to limit batch effects. Applied Biosystems Transcriptomic Analysis Console (TAC) software was used to calculate fold-changes in gene expression relative to the untreated control cells. Values were considered statistically significant for p values <0.05.

Statistical analysis. One-way Anova was used to determine statistical significance of groups of three or more and a post-hoc Tukey's multiple comparisons test was used for multiple comparisons. Two-tailed, paired student's t tests were used to determine statistical significance of pairs. Significance is reported as follows: P≤0.05: *, P≤0.01: **, and P≤0.001: ***.

Assay for colorectal cancer patients who will benefit from this therapy. A patient's genomic signatures and serum or plasma cytokine profiles may be useful in the prediction of response to the immune stimulatory effects of maleimide derivatives. MSI+ or MSS patients with phenotypic “cold” tumors or “immune deserts with limited immune cell infiltration, could also benefit from this therapy as this therapy increases T cell tumor-infiltration.

Additional assays for colorectal cancer patients who will benefit from this therapy are described by Bosch et al., Molecular tests for colorectal cancer screening. Clin. Colorectal Cancer, 10(1), 8-23 (Mar. 1, 2011).

Assay for COVID-19 to select for patients who will benefit from this treatment. A patient's genomic signatures and serum or plasma cytokine profiles may be useful in the prediction of response to the anti-viral effects of maleimide derivatives.

Additional assays for COVID-19 patients who will benefit from this therapy are described by Bosch et al., Molecular tests for colorectal cancer screening. Clin. Colorectal Cancer, 10(1), 8-23 (Mar. 1, 2011).

Additional assays for COVID-19 patients who will benefit from this therapy are described by The United States Centers for Disease Control.

The following EXAMPLES are provided to illustrate the invention and shall not limit the scope of the invention.

### Example 1

Inhibition of GSK-3β with the Small Molecule 9-ING-41 Stimulates Natural Killer and T Cell Activity, Reduces Vascular Endothelial Growth Factor Levels, and Induces Cancer Cell Death

GSK-3β inhibition attenuates the systemic inflammatory response (SIR) in models of sepsis and ischaemia/reperfusion injury by modulating NF-kB-induced inflammatory response. See Dugo et al., Crit. Care Med., 33, 1903-12 (2005); Martin et al., Nature Immunol., 6, 777-784 (2005); Dugo et al., Shock, 25, 485-91 (2006); and Cuzzocrea et al., Intensive Care Med., 33, 880-893 (2007). These findings have potential implication to systemic inflammatory response and the frequent disseminated intravascular vascular coagulopathy observed during the infection with SARS-CoV2. GSK-3β inhibition also decreased mRNA expression of IL-1β, IL-6, and inducible NO synthase (iNOS) in a model of lipopolysaccharide (LPS) mediated inflammation. See Huang et al., Immunology, 128, e275-86 (2009).

The potent and selective ATP-competitive GSK-3β inhibitor 9-ING-41 has advanced to the clinic with preliminary results from ongoing clinical trials involving patients with advanced malignancies demonstrating an excellent safety profile devoid of myelosuppressive and immunosuppressive effects.

In an immune cell-killing co-culture assay, a significant increase in natural killer (NK-92) cell and T cell (TALL-104) killing of the colorectal cancer cells occurred in response to treatment with 9-ING-41 as compared to controls without drug treatment.

A panel of human colorectal cell lines was chosen to provide a varied mutational background (TP53, KRAS, BRAF, TRK, APC, PIK3CA).

Cells were treated with 9-ING-41, a selective and potent small molecule inhibitor of GSK-3β, at doses up to 100 μM for seventy-two hours to determine IC₁₀, IC₃₀, IC₅₀, and IC₇₀. Cytokine, chemokine, and growth factor levels were analyzed in the tumor cell culture supernatants after treatment at IC₁₀, IC₃₀, IC₅₀, and IC₇₀ for forty-eight hours using a Luminex 200 multiplexing instrument.

Co-culture experiments were conducted with GFP+ SW480 colorectal cancer cells and either NK-92 natural killer cells or TALL-104 T cells at various effector/target ratios in a 48-well plate, in the presence or absence of 9-ING-41.

The inventors observed a stimulation of Natural Killer activity by 9-ING-41 treatment at IC₅₀ dose. The inventors co-cultured green fluorescent SW480 tumor cells with NK-92 natural killer cells at a 1:1 effector target cell ratio (E:T) for twenty-four hours and imaged by fluorescence microscopy. Ethidium homodimer was used to visualize dead cells. The inventors obtained photographic images of the co-cultured cells.

Overall cytokine levels showed a decreasing trend in response to increasing doses of 9-ING-41. Among the most prominently decreased growth factors in the profile was VEGF.

Follow-up experiments compared the effect of pre-treating either the effector or the target cell population with 9-ING-41 before the co-culture experiment was started. Pre-treatment of the target tumor cells, but not the effector immune cells, bolstered cell-killing, implying that the drug is sensitizing the tumor cells to killing by the immune cells.

The inventors saw an increase in the expression of chemokine CXCL14 (BRAK) in the tumor cell culture supernatant with increasing doses of 9-ING-41. BRAK is known to stimulate activated NK cell migration and could have a beneficial therapeutic effect by increasing NK cell migration into the tumor microenvironment.

The inventors saw a decrease in macrophage colony-stimulating factor (M-CSF) which, along with VEGF, has been associated with recruitment of tumor-associated macrophages.

The compound 9-ING-41 is not associated with myelosuppression of any degree. There has been no evidence of increased infection or any opportunistic/unusual infections even in patients with extensive prior cytotoxic therapy. A complete response was documented in a patient with BRAF V600E metastatic melanoma previously treated with dabrafenib/trametinib and nivolumab®, including resolution of brain metastases. Evidence of durable responses and prolonged treatment duration among patients with pancreatic cancer receiving 9-ING-41 plus gemcitabine/nab-paclitaxel and endometrial cancer receiving 9-ING-41 plus carboplatin/paclitaxel has supported the recent activation of a phase 2 study investigating the former regimen in the front-line treatment of metastatic pancreatic cancer with other Phase 2 studies in preparation.

This anti-fibrotic activity supported the clinical development and ongoing clinical trial of 9-ING-41 in patients with advanced myelofibrosis (NCT04218071). Considering the likely high global burden of fibrotic lung disease following the pandemic, GSK-3β inhibition with 9-ING-41 could also have a favorable impact in reducing the lung sequelae from COVID-19 infection.

Based on 9-ING-41's established safety profile and pre-clinical potent anti-fibrotic activity aligned with the robust preclinical rationale for GSK-3β blockade, this GSK-3β inhibitor merits urgent consideration as an investigational therapy for patients with clinically significant COVID-19 infection.

### Example 2

### Small-Molecule Inhibition of Glycogen Synthase Kinase-3 Increases Efficacy of αPD-L1 Therapy in an Immunocompetent Murine Model of Microsatellite Stable (MSS) Colorectal Cancer

The compound 9-lNG-41 boosts immune cell-mediated tumor cell-killing in a co-culture model. A co-culture of GFP+ SW480 tumor cells and Green CMFDA-labeled TALL-104 T cells treated with 9-ING-41 lead to an increase in tumor cell death after twenty-four hours. Doses chosen were significantly less than the 24-hour ICsos calculated for all cell lines evaluated in the co-culture. Limited tumor cell death occurred in SW480 monocultures treated with drug only. See FIG. 3(A). In the co-culture with tumor and immune cells only, in the absence of drug, the inventors noted that the percentage of dead cells out of total cells was approximately 27%, after normalization. Normalization was carried out by subtracting the percentage of cell death due to drug or vehicle control (DMSO) only from the percentage of dead cells observed in the co-culture treated with drug. The inventors noticed a dose-dependent increase in the amount of TALL-104 cell-mediated SW480 cell-killing after treatment with 9-ING-41. See FIG. 3(B). Co-cultures treated with 5 μM 9-ING-41 had an average of 33% dead cells, while co-cultures treated with 10 μM of 9-ING-41 had an average of 37% dead cells.

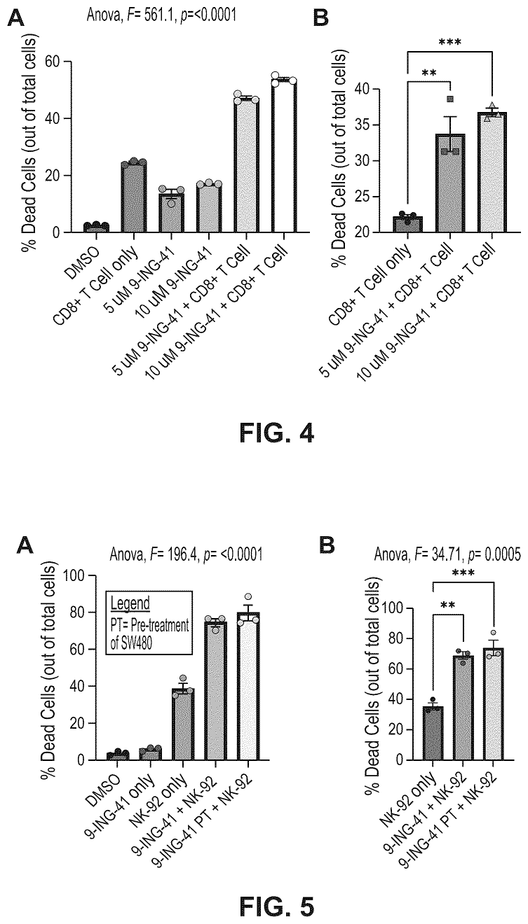

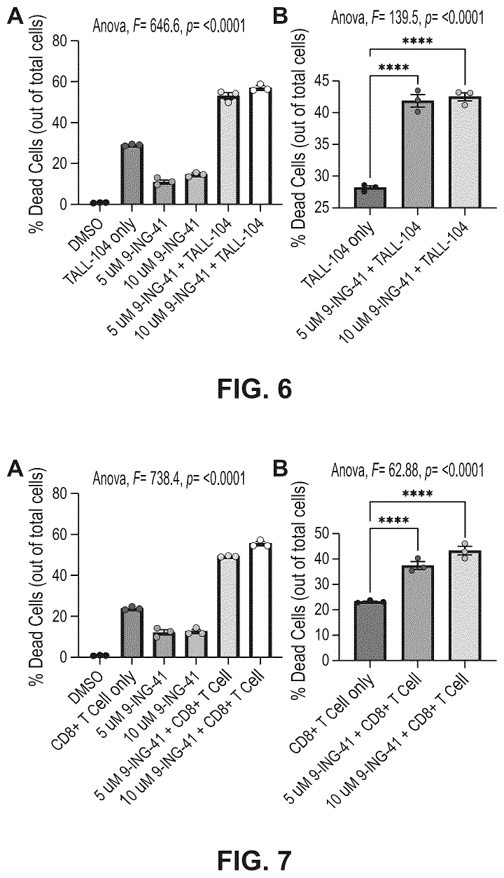

TALL-104 cells are a human leukemic T cell line. The relevancy of these results was next determined using normal T cells. Healthy CD8+ T cells were isolated from a donor blood sample consistent with an IRB-approved protocol. A co-culture of GFP+ SW480 tumor cells and Green CM FDA-labeled CD8+ donor-derived healthy T cells was then treated with 9-ING-41 and the % of dead cells out of total cells was quantified after twenty-four hours. Limited tumor cell death occurred in SW480 monocultures treated with drug only. See FIG. 3(A). The data was then normalized, as described earlier. Even more impressive tumor cell death occurred in the co-cultures treated with 9-ING-41. See FIG. 4(C).

Pre-treatment with 9-ING-41 sensitizes SW480 tumor cells to immune cell-mediated cell killing. To determine if the increased amount of immune-cell mediated tumor cell-killing was due to the drug's impact on the tumor cells or the immune cells, the inventors next pre-treated tumor cells with 9-ING-41 for twenty-four hours before the co-culture with immune cells began. Pre-treatment with 9-ING-41 sensitized SW480 tumor cells to TALL-104 cell-mediated cell killing. The raw percentages of cell death were used to normalize the data. See FIG. 3(A) and FIG. 3(B). The inventors confirmed these co-culture results using healthy, CD8+ T cells instead of TALL-104 cells. Similar results occurred with the CD8+ T cells where 9-ING-41 pre-treatment of tumor cells lead to a statistically significant increase in tumor cell death after twenty-four hours of co-culture. See FIG. 7(A) and FIG. 7(B).

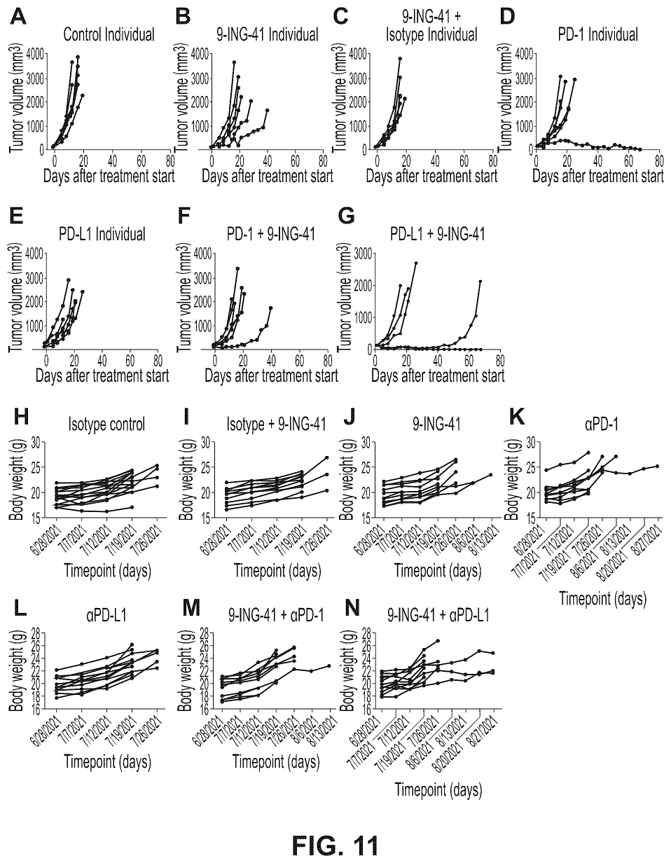

To determine the relevancy of these results to other cytotoxic lymphocytes, the co-culture was repeated using a natural killer cell line, NK-92. See FIG. 11. An average baseline amount of NK-92-mediated tumor cell killing of about 39% occurred in the absence of drug. See FIG. 11. After normalization statistically significant increase occurred in dead cells, as compared to total cells.

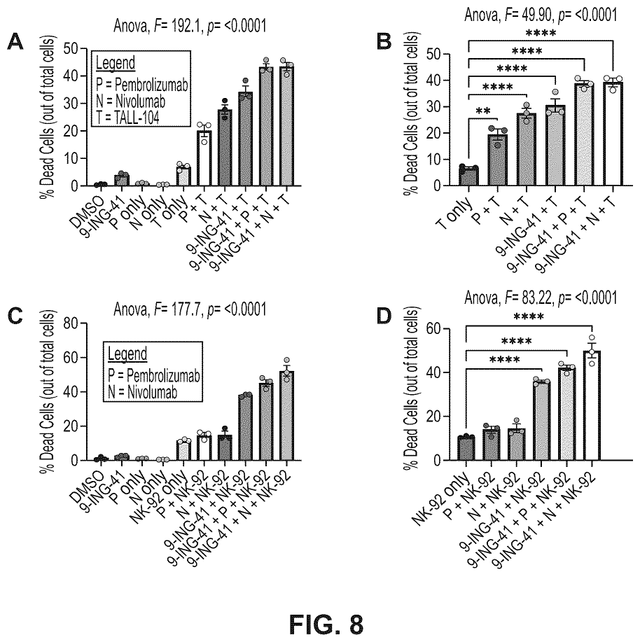

9-ING-41 increases tumor cell death in combination with αPD-1 therapy in a co-culture of HCT-116 colorectal cancer cells and immune cells. The inventors were interested in the applicability of GS3K-3 inhibitors such as 9-ING-41 in combination with immune checkpoint blockade. The inventors next performed co-cultures of immune cells (TALL-104, NK-92) and tumor cells (HCT-116) in the presence or absence of 9-ING-41, as well as either pembrolizumab® or nivolumab®, two humanized monoclonal antibodies against PD-1. The inventors evaluated HCT-116 cells in this co-culture model to determine if the 9-ING-41-mediated increase in immune cell-mediated SW480 cell killing could be reproduced in another colorectal cancer cell line. The co-culture of HCT-116 and TALL-104 cells showed similar trends as earlier in terms of tumor cell death post-treatment with 9-ING-41 as well as post-incubation with the T cell line only. The inventors noted an increased amount of TALL-104-mediated tumor cell killing when either pembrolizumab® or nivolumab® was added to the co-culture, as compared to TALL-104 cells alone. See FIG. 8(A). A slight increase in cell death occurred with pembrolizumab®+TALL-104 as compared to nivolumab®+TALL-104. The 9-ING-41+TALL-104 culture condition (without αPD-1 therapy) showed an even greater amount of cell death after twenty-four hours of co-culture. The triple therapy groups (TALL-104+9-ING-41+pembrolizumab®/nivolumab®) showed the most impressive results See FIG. 8(B).

The inventors next determined if these results were applicable to the natural killer cell line, NK-92. Both pembrolizumab® and nivolumab® in combination with NK-92 offered limited benefit as compared to NK-92 alone, although not statistically significant. See FIG. 8(C). 9-ING-41+NK-92 provided a significant benefit over the combination αPD-1 therapy+NK-92 only groups. The triple therapy groups showed the greatest amount of cell death after the 24-hour co-culture, where the triple therapy group including nivolumab® was the most impressive. See FIG. 8(D).

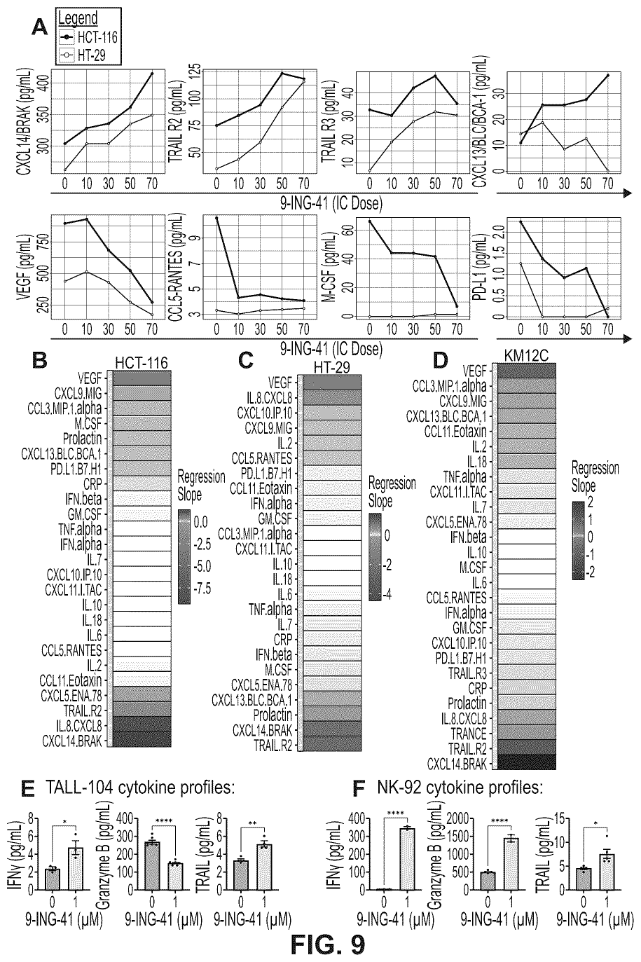

9-ING-41 increases secreted cytotoxic proteins IFN-v, Granzyme B, and TRAIL in immune cells. The inventors previously found that 9-ING-41 treatment of human colorectal cell lines (HCT-116, HT-29, KM12C) with varied mutational modified cytokine, chemokine, and growth factor profiles. Huntington, Louie, Zhou, & EI-Deiry, Oncotarget, Vol. 12, No. 20 (2021). Here, the inventors determined how 9-ING-41 treatment impacts the immune cell cytokinome. TALL-104, healthy donor-derived CD8+T, and NK-92 cells were treated with 1 μM 9-ING-41 for forty-eight hours before cell culture supernatant was collected for cytokine profile analysis. TALL-104 cells treated with 9-ING-41 showed increases in IFN-γ, granzyme B, and TRAIL concentrations, as measured in picogram per milliliter. See FIG. 9(A). Healthy, donor-derived CD8+ T cells treated with 9-ING-41 also showed increases in all three cytotoxic molecules analyzed. See FIG. 9(B). NK-92 cells treated with 9-ING-41 showed increases in IFN-γ and TRAIL, but showed decreases in the concentration of secreted granzyme B. See FIG. 9(C).

9-ING-41 treatment decreases survival factor expression while increasing PD-L1 levels in colorectal cancer cell lines. To elucidate the mechanism behind the differential effects of 9-ING-41 on either colorectal cancer or immune cells in co-culture and cytokine profiling assays, the inventors performed Western blot analyses on tumor and immune cells treated with 9-ING-41 over a 72-hour time course.

Treating cells from colorectal cancer cell lines with 1 μM 9-ING-41 decreases Surivin, NF-κB p65, and IκBα while increasing PD-L1 levels in, 9-ING-41 treatment of immune cells increases NIK. Western blot analysis of HCT-116 and HT-29 colorectal cancer cells for expression of indicated proteins after increasing durations of 9-ING-41 treatment (0-72 hours). Western blot analysis of TALL-104 T cells for expression of indicated proteins after increasing durations of 9-ING-41 treatment (0-72 hours). Analysis of Mcl-1 expression in HCT-116 colorectal cancer cells and NK-92 natural killer cells after increasing durations of 9-ING-41 treatment.

In HCT-116 cells, little to no cPARP occurred until the 48-hour timepoint No changes occurred in IKKR, which is a kinase that mediates phosphorylation of IKB. The inventors observed decreases in Survivin, NF-κB-inducing kinase (NIK), NF-κB, IKBα, pIKBα, and Bcl-2 all by the 72-hour timepoint. The inventors observed increases in PD-L1 expression as the treatment duration increased. In HT-29 cells, the inventors noted similar trends where Survivin, NF-KB, IKBα, pIKBα, and Bcl-2 all decreased by the 72-hour timepoint. IKKβ and Nik did not change. PD-L1 increased as treatment duration increased.

When probing for the same proteins in TALL-104 cell lysates, the inventors saw opposing trends to those observed in the tumor cells. In TALL-104 cells, no significant changes in NF-KB, IKK β, IKBα, pIKBα, and Bcl-2 was observed as treatment duration increased. The inventors noted surprising increases in Survivin and NIK. The inventors predictably observed decreased in PD-1 expression. Because of the differential impact of 9-ING-41 on tumor and immune cells as shown via Western blot, the inventors compared the levels of another survival protein Mcl-1 in HCT-116 cells as compared to NK-92 cells. In HCT-116 cells, marked decreases in protein expression occurred by the 24-hour timepoint. The inventors did not observe a significant decrease in Mcl-1 protein expression through the 48-hour endpoint.

The inventors did not focus on 9-ING-41's effects on β-catenin because colon cancers often harbor mutations in β-catenin or adenomatous polyposis coli (APC), thus nullifying any impact GSK-3 inhibition would have on β-catenin expression. For example, HCT116 cells are heterozygous for β-catenin, harboring one wild type allele and one mutant allele with inactivation of one of the residues (SER45) phosphorylated by GSK-3β that is frequently mutated in tumors. Kaler, Augenlicht, & Klampfer, PLoS ONE, 7, e45462 (2012). HT-29, KM12C, and SW480 cells harbor APC mutations. See Rowan et al., Proceedings of the National Academy of Sciences, 97, 3352 (2000).

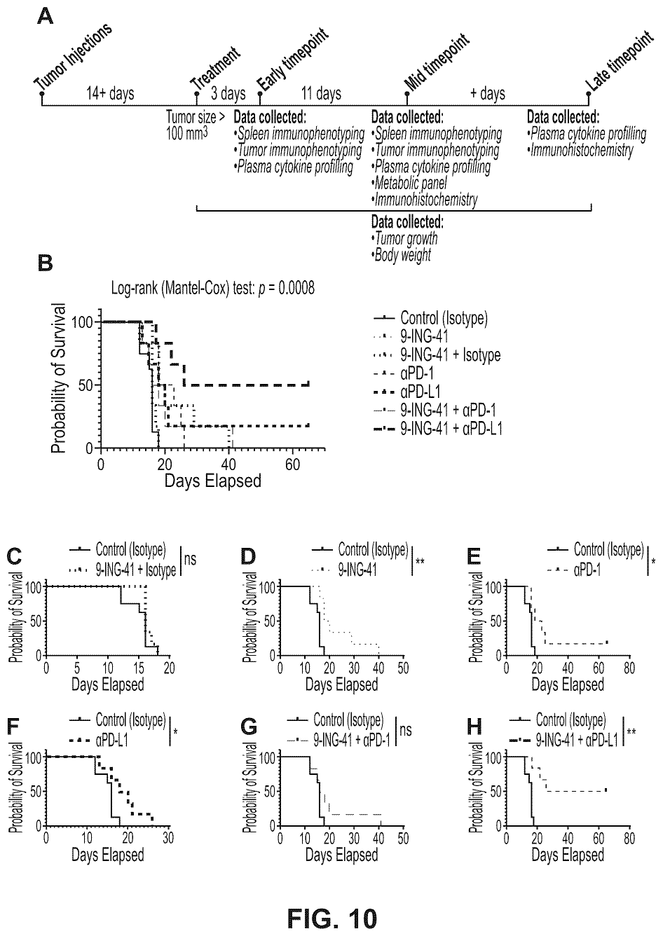

Flow cytometric analysis of cell surface markers of tumor and immune cells treated with 9-ING-41 in vitro shows differential expression of HLA-E, PD-L1, ICAM-1, DR5, NKG2A, TRAIL, and LAG-3. To further understand the differential 9-ING-41-mediated impacts on protein expression, the inventors treated tumor and immune cells in vitro with 9-ING-41, then performed a flow cytometric analysis for expression of cell surface markers. A statistically significant increase was observed in non-classical MHC class I molecule HLA-E and checkpoint ligand PD-L1 surface expression via flow cytometry in both tumor cell lines (HCT-116, HT-29) post-treatment with 9-ING-41. See FIG. 10(A-B). The inventors also observed a significant increase in ICAM-1 expression in HCT-116 colorectal cancer cells post-treatment, which binds leukocyte function associated antigen-1 (LFA-1) to aid in immune cell recruitment. In HCT-116 cells, DR5 expression significant decreased post-treatment. A similar trend occurred in HT-29 cells, although not statistically significant. In natural killer cells (NK-92) treated with 9-ING-41 for forty-eight hours, the inventors observed statistically significant decreases of checkpoint molecule NKG2A and increases in apoptosis-inducing ligand TRAIL and LAG3 surface expression. FIG. 10(C). In the TALL-104 cells, TRAIL expression significant decreased, as compared to DMSO treatment alone. FIG. 10(D).

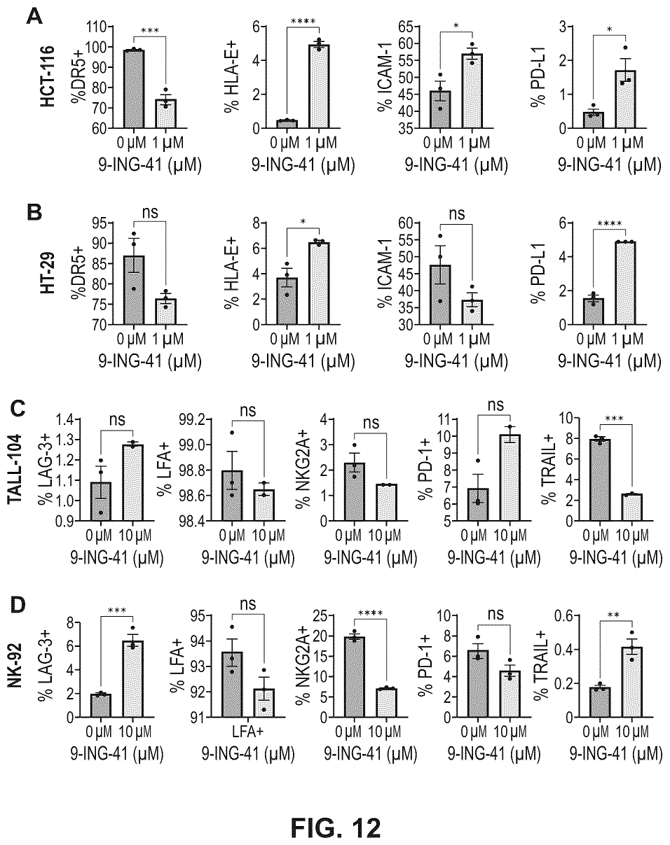

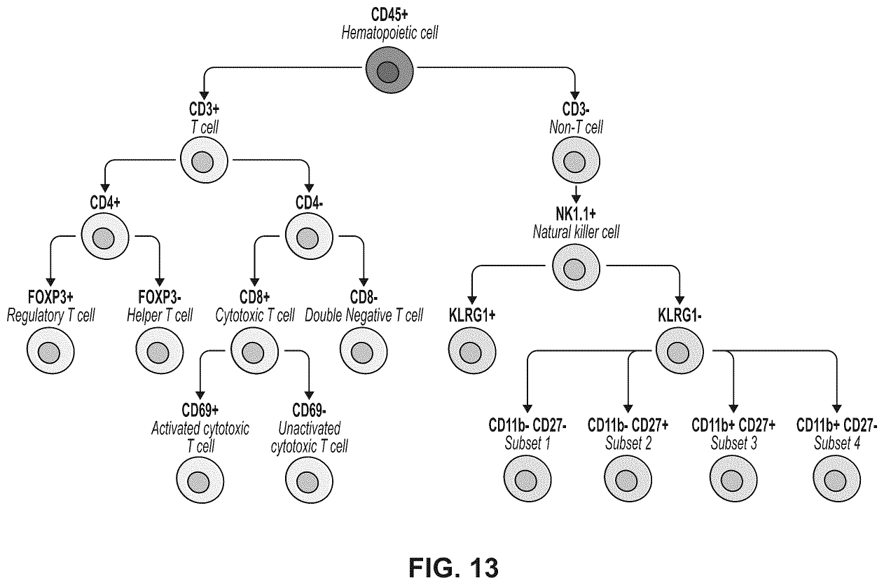

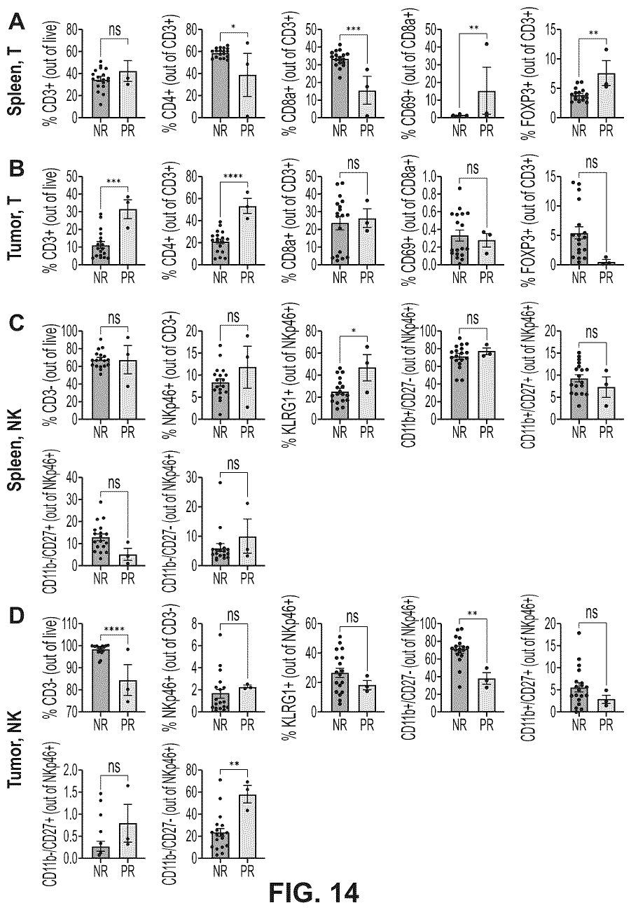

9-ING-41 significantly prolongs survival in combination with αPD-L1 therapy in a syngeneic microsatellite stable colorectal cancer murine model. To evaluate the potential for 9-ING-41 to increase efficacy of immune checkpoint blockade, the inventors used a syngeneic murine colon carcinoma BALB/c murine model with a microsatellite stable (MSS) cell line CT-26. See FIG. 12(A). In this microsatellite stable colorectal cancer model, a significantly improved survival curve occurred in the 9-ING-41 and αPD-L1 combination therapy group. See FIG. 12(B). The inventors also observed statistically significant improved survival in the 9-ING-41, αPD-1, and αPD-L1 alone groups as compared to control. The most sustained response occurred in the 9-ING-41 and αPD-L1 combination therapy group.

One mouse had a durable response to αPD-1 therapy alone. Two mice had durable responses to combination 9-ING-41 and αPD-L1 therapy. Body weights of mice did not differ significantly regardless of treatment group.

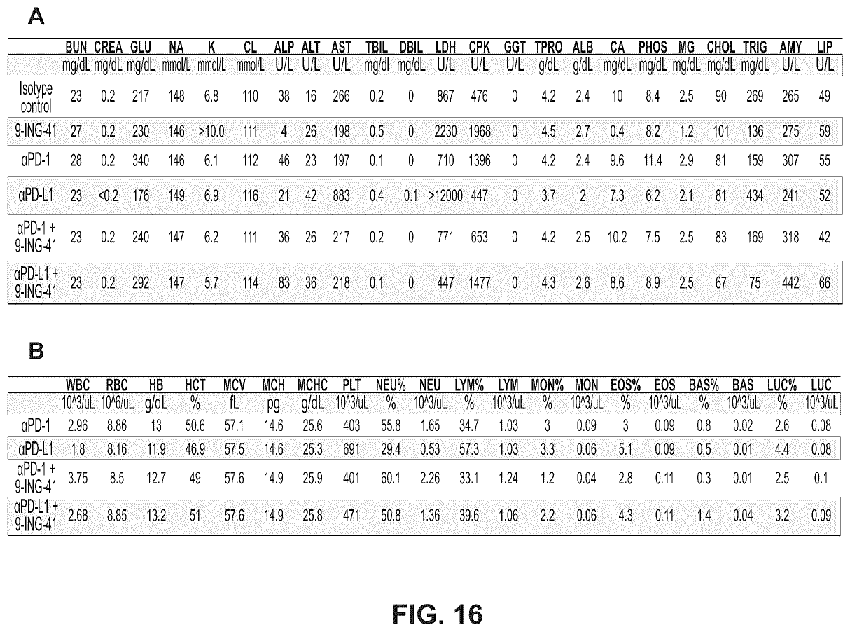

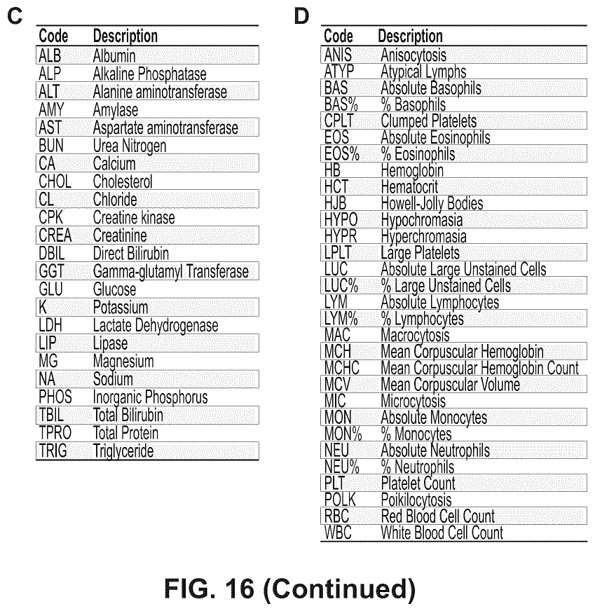

Mice did not show evidence of significant treatment-related toxicity on complete blood count or serum chemistry analysis. See FIG. 16. Both 9-ING-41 individual treatment group and dual treatment groups maintained normal renal function as evidenced by normal blood urea nitrogen (BUN) and creatinine and were free of significant electrolyte perturbations. Liver function tests did not reveal any evidence of liver toxicity and the dual treatment mice did not have any elevations of AST, ALT, or bilirubin. As can be expected in mice with significant tumor burdens, mice across treatment groups had decreased albumin levels and evidence of mild marrow hypoplasia resulting in mild anemia, and lower white blood cell and platelet counts. This effect was independent of treatment group and likely related to tumor burden at the time of sacrifice.

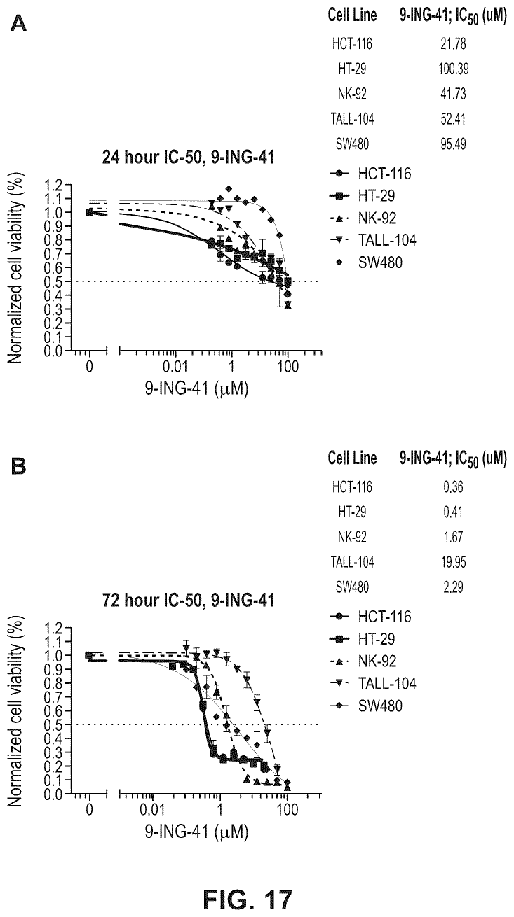

Partial responders have lower percentages of splenic CD4+ T cells and splenic CD8+ T cells, and increased percentages of CD69+ activated T cells, FOXP3+ regulatory T cells, CD3+ TILS, CD4+ ILS, and splenic KLRG1+ mature NK cells. To begin to analyze the changes in immune subsets in response to therapy, the inventors used multi-color flow cytometry to characterize the natural killer (NK) and T cell populations. See FIG. 17. Compared to non-responders in any treatment group, responders 14-days post-treatment had statistically significantly lower levels of splenic CD4+ and CD8+ T cells and had increased percentages of CD69+ activated T cells and FOXP3+ regulatory T cells. FIG. 14(A). Meanwhile, partial responders had increased percentages of tumor infiltrating CD3+ and CD4+ T cells. FIG. 14(B). The inventors also observed that partial responders had increased percentages of splenic KLRG1+ mature NK cells and tumor-infiltrating CD11b−/CD27− immature NK cells, and decreased percentages of CD11b+/CD27− activated NK cells 14-days post-treatment initiation. See FIG. 14. No striking differences between non-responders and partial responders in the splenic natural killer cell subsets (CD11b−/CD27−, CD11b−/CD27+, CD11b+/CD27+, CD11b+CD27−) were observed. See FIG. 14. The inventors observed significant differences between non-responders and partial responders in the tumor-infiltrating natural killer cell subsets. Partial responders had a greater proportion of immature (CD11b−/CD27−) NK cells and a lower proportion of mature (CD11b+CD27−) NK cells 14-days post-treatment initiation.

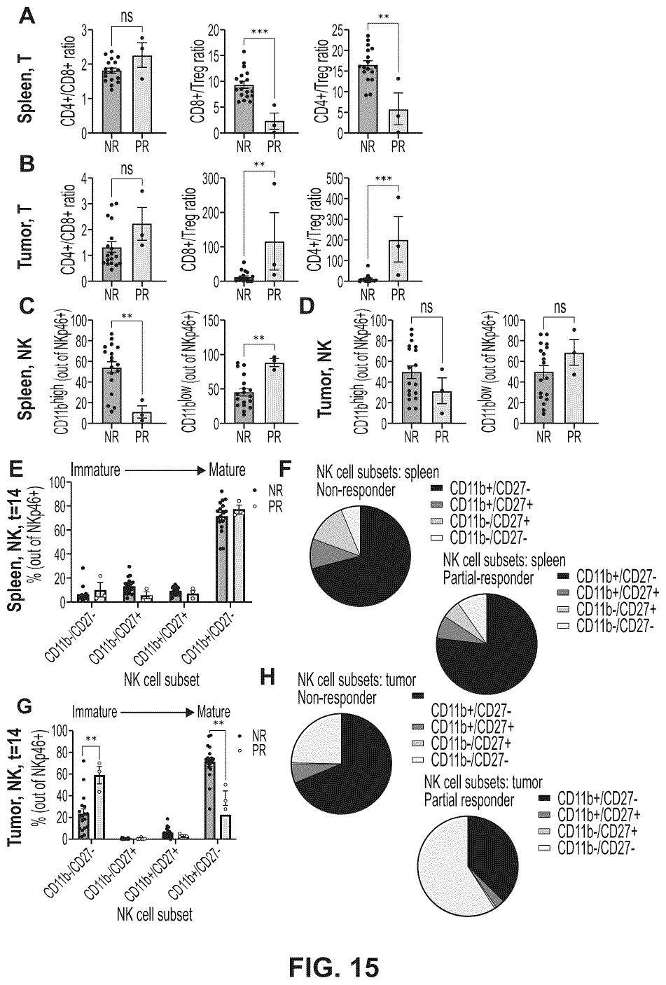

Splenic and intra-tumoral T cell ratios differ between non-responders and partial responders. When comparing the T cell ratios, compared to non-responders, partial responders had a lower splenic CD8+/Treg and CD4+/Treg ratio. See FIG. 15A). Additionally, partial responders had a higher intra-tumoral CD8+/Treg and CD4+/Treg ratio. See FIG. 15(B). The inventors noted a decreased percentage of splenic CD11bhigh and an increased percentage of splenic CD11 blow NK cells in partial responders compared to non-responders. FIG. 15(C). The inventors observed similar trends in the tumor-infiltrating lymphocyte (TIL) NK cell population, but the differences were not statistically significant. FIG. 15(D).

Responders have lower serum concentrations of BAFF, CCL7, CCL12, VEGF, VEGFR2, and CCL21 and higher serum concentrations of CCL4, TWEAK, GM-CSF, CCL22, and IL-12p70 compared to non-responders. The inventors next analyzed murine serum samples for cytokine profiles and noted interesting trends between partial, complete, and non-responders. Complete and partial responders were more likely to have lower serum concentrations of BAFF, CCL7, CCL12, VEGF, VEGFR2, and CCL21 compared to non-responders. Complete and partial responders had higher serum concentrations of CCL4, TWEAK, GM-CSF, CCL22, and IL-12p70 compared to non-responders.

### List of Embodiments

Specific compositions and methods of GSK-3 inhibition as a therapeutic approach against SARs CoV2. The scope of the invention should be defined solely by the claims. A person having ordinary skill in the biomedical art will interpret all claim terms in the broadest possible manner consistent with the context and the spirit of the disclosure. The detailed description in this specification is illustrative and not restrictive or exhaustive. This invention is not limited to the particular methodology, protocols, and reagents described in this specification and can vary in practice. When the specification or claims recite ordered steps or functions, alternative embodiments might perform their functions in a different order or substantially concurrently. Other equivalents and modifications besides those already described are possible without departing from the inventive concepts described in this specification, as persons having ordinary skill in the biomedical art recognize.

All patents and publications cited throughout this specification are incorporated by reference to disclose and describe the materials and methods used with the technologies described in this specification. The patents and publications are provided solely for their disclosure before the filing date of this specification. All statements about the patents and publications' disclosures and publication dates are from the inventors' information and belief. The inventors make no admission about the correctness of the contents or dates of these documents. Should there be a discrepancy between a date provided in this specification and the actual publication date, then the actual publication date shall control. The inventors may antedate such disclosure because of prior invention or another reason. Should there be a discrepancy between the scientific or technical teaching of a previous patent or publication and this specification, then the teaching of this specification and these claims shall control.

When the specification provides a range of values, each intervening value between the upper and lower limit of that range is within the range of values unless the context dictates otherwise.

### CITATION LIST

A person having ordinary skill in the biomedical art can use these patents, patent applications, and scientific references as guidance to predictable results when making and using the invention.

### Patent Literature

International Pat. Publ. WO 2020/123813 A1 (Actuate Therapeutics, Inc.), Immunohistochemical staining for GSK-3, published Jun. 18, 2020.

### Non-Patent Literature

Augello et al., The role of GSK-3 in cancer immunotherapy: GSK-3 inhibitors as a new frontier in cancer treatment. Cells, 9 (2020).
Bendardaf et al., VEGF-1 expression in colorectal cancer is associated with disease localization, stage, and long-term disease-specific survival. Anticancer Res. 28, 3865-3870 (2008).
Carneiro et al., Phase I study of 9-ING-41, a small molecule selective glycogen synthase kinase-3 beta (GSK-3(3) inhibitor, as a single agent and combined with chemotherapy, in patients with refractory tumors. J. Clin. Oncol., 38(Suppl.): abstr 3507 (2020). This document presented the initial data from an ongoing phase I/II clinical trial of 9-ING-41 administered as monotherapy or combined with several chemotherapy regimens at the annual meeting of the American Society of Clinical Oncology (NCT03678883). The study has enrolled over 200 patients with advanced malignancies and completed cohorts with monotherapy and combination with various standard chemotherapy regimens without attributable Grade 3 or 4 serious adverse events.
Cuzzocrea et al., Glycogen synthase kinase-3beta inhibition attenuates the development of ischaemia/reperfusion injury of the gut. Intensive Care Med. 33, 880-893 (2007).
Ding et al., Glycogen synthase kinase-3 inhibition sensitizes pancreatic cancer cells to chemotherapy by abrogating the TopBP1/ATR-mediated DNA damage response. Clin. Cancer Res., 25, 6452-6462 (2019). The compound 9-ING-41 reduced pulmonary fibrosis and improved pulmonary function in models of TGF-β.
Ducloyer et al., Complete post-mortem data in a fatal case of COVID-19: clinical, radiological and pathological correlations. Int. J. Legal Med. (2020). Autopsy studies demonstrated marked fibrotic lung disease in patients who suffered from the COVID-19.
Dugo et al., Glycogen synthase kinase-3beta inhibitors protect against the organ injury and dysfunction caused by hemorrhage and resuscitation. Shock, 25, 485-491 (2006).
Dugo et al., GSK-3beta inhibitors attenuate the organ injury/dysfunction caused by endotoxemia in the rat. Crit. Care Med., 33, 1903-1912 (2005).
Gaisina, et al. From a natural product lead to the identification of potent and selective benzofuran-3-yl-(indol-3-yl)maleimides as glycogen synthase kinase 3beta inhibitors that suppress proliferation and survival of pancreatic cancer cells. J. Med. Chem., 52(7), 1853-63 (Apr. 9, 2009).
Gavalas et al., VEGF directly suppresses activation of T cells from ascites secondary to ovarian cancer via VEGF receptor type 2. British Journal of Cancer, 107, 1869-1875 (2012).
Gupta, Sinha, & Paul, The impact of microsatellite stability status in colorectal cancer. Current Problems in Cancer, 42, 548-559 (2018).
Hassounah et al., Identification and characterization of an alternative cancer-derived PD-L1 splice variant. Cancer Immunol. Immunother., 68, 407-420 (2019).
Hilliard et al., Glycogen synthase kinase 3beta inhibitors induce apoptosis in ovarian cancer cells and inhibit in-vivo tumor growth. Anticancer Drugs, 22, 978-85 (2011). Results from in vitro and xenograft models of ovarian cancer demonstrated that 9-ING-41 was more active than lithium and other ATP-competitive inhibitors such as SB216763, the compound used in the experiments showing the enhancement of T cell function. Lithium has a narrow therapeutic index and is significant less potent than other small-molecule GSK-3β inhibitors in clinical development.
Huang et al., Glycogen synthase kinase-3 negatively regulates anti-inflammatory interleukin-10 for lipopolysaccharide-induced iNOS/NO biosynthesis and RANTES production in microglial cells. Immunology, 128, e275-86 (2009).
Huntington, Louie, Zhou, & El-Deiry, A high-throughput customized cytokinome screen of colon cancer cell responses to small-molecule oncology drugs. Oncotarget, Vol. 12, No. 20 (2021).
Jeffers et al., Glycogen synthase kinase-3beta inhibition with 9-ING-41 attenuates the progression of pulmonary fibrosis. Sci. Rep. 9, 18925 (2019). The compound 9-ING-41 reduced pulmonary fibrosis and improved pulmonary function in models of bleomycin pulmonary fibrosis.
Jellestad et al., Inhibition of glycogen synthase kinase (GSK)-3-beta improves liver microcirculation and hepatocellular function after hemorrhagic shock. Eur. J. Pharmacol., 724, 175-84 (2014). Animal models of hemorrhagic shock show that GSK-3β inhibition dampens liver and renal dysfunction by upregulation of anti-inflammatory IL-10 and down-regulation of IL-12p40 and IL-6, a cytokine implicated in the cytokine release syndrome observed in patients with severe SARS-CoV2.
Kaler, Augenlicht, & Klampfer, Activating mutations in β-catenin in colon cancer cells alter their interaction with macrophages; the role of Snail. PLoS ONE, 7, e45462 (2012).
Karmali et al., GSK-3β. inhibitor, 9-ING-41, reduces cell viability and halts proliferation of B-cell lymphoma cell lines as a single agent and in combination with novel agents. Oncotarget, Vol. 8, No. 70 (2017).
Keith et al., GSK-3 beta: A bifunctional role in cell death pathways. International Journal of Cell Biology (2012).
Kelsey et al., GSK-311 inhibition by small molecule 9-ING-41 decreases VEGF and other cytokines, and boosts NK and T cell-mediated killing of colorectal tumor cells. AACR 2021 #2676.
Kuroki, et al. 9-ING-41, a small molecule inhibitor of GSK-3beta, potentiates the effects of anticancer therapeutics in bladder cancer. Sci Rep., 9(1), 19977 (Dec. 27, 2019).
Lee et al., Crosstalk between CCL7 and CCR3 promotes metastasis of colon cancer cells via ERK-JNK signaling pathways. Oncotarget, 7, 36842-36853 (2016).
Li et al., Oncogenic K-ras stimulates Wnt signaling in colon cancer through inhibition of GSK-3beta. Gastroenterology, 128, 1907-1918 (2005).
Li, Sun, Tao, & Wang, The CCL21/CCR7 pathway plays a key role in human colon cancer metastasis through regulation of matrix metalloproteinase-9. Dig. Liver Dis. 43, 40-47 (2011).
Martin et al., Toll-like receptor-mediated cytokine production is differentially regulated by glycogen synthase kinase 3. Nature Immunol., 6, 777-784 (2005).
Medunjanin et al., GSK-3β controls NF-kappaB activity via IKKy/NEMO. Sci. Rep. 6, 38553 (2016).
Middha et al., Majority of B2M-mutant and -deficient colorectal carcinomas achieve clinical benefit from immune checkpoint inhibitor therapy and are microsatellite instability-high. JCO Precis Oncol., 3 (2019).
Moeller et al., A functional role for CD28 costimulation in tumor recognition by single-chain receptor-modified T cells. Cancer Gene Ther., 11, 371-379 (2004).
Nakayama et al., Involvement of TWEAK in interferon gamma-stimulated monocyte cytotoxicity. J. Exp. Med. 192, 1373-1380 (2000).
Rihacek et al., B-Cell activating factor as a cancer biomarker and its implications in cancer-related cachexia. BioMed Research International 2015, pages 792187-792187.
Rowan et al., APC mutations in sporadic colorectal tumors: A mutational “hotspot” and interdependence of the “two hits”. Proceedings of the National Academy of Sciences, 97, 3352 (2000).
Rudd, Chanthong, & Taylor, Small molecule inhibition of GSK-3 specifically inhibits the transcription of inhibitory co-receptor LAG-3 for enhanced anti-tumor immunity. Cell Reports, 30, 2075-2082.e2074 (2020).
Rudd, GSK-3 inhibition as a therapeutic approach against SARs CoV2: Dual benefit of inhibiting viral replication while potentiating the immune response. Front. Immunol. 11:1638 (2020). The manuscript describes the GSK-3-mediated phosphorylation of key serine residues in SARS-CoV2 nucleocapsid proteins essential for viral replication. Dr. Rudd discusses the therapeutic potential of specific GSK-3 inhibitors (i.e., SB216763, tideglusib) and proposes lithium as a GSK-3β inhibitor to consider for clinical trials, an available oral drug with known toxicity profile.
Sahin et al., Glycogen synthase kinase-3 beta inhibitors as novel cancer treatments and modulators of antitumor immune responses. Cancer Biol. Ther. 20, 1047-56 (2019). Lithium has a narrow therapeutic index and is significant less potent than other small-molecule GSK-3β inhibitors in clinical development.
Saitoh et al., TWEAK induces NF-kappaB2 p100 processing and long-lasting NF-kappaB activation. J. Biol. Chem., 278, 36005-36012 (2003).
Schwensen et al., Fatal pulmonary fibrosis: a post-COVID-19 autopsy case. J Clin Pathol. (2020). Autopsy studies demonstrated marked fibrotic lung disease in patients who suffered from the COVID-19.
Spruch et al., Does lithium deserve a place in the treatment against COVID-19? A preliminary observational study in six patients, case report. Frontiers in Pharmacology, 11, 557629 (2020).
Starnes et al., The chemokine CXCL14 (BRAK) stimulates activated NK cell migration: Implications for the downregulation of CXCL14 in malignancy. Experimental Hematology, 34, 1101-1105 (2006).
Steding et al., The role of interleukin-12 on modulating myeloid-derived suppressor cells, increasing overall survival and reducing metastasis. Immunology, 133, 221-238 (2011).
Sung et al., Global cancer statistics 2020: GLOBOCAN estimates of incidence and mortality worldwide for 36 cancers in 185 countries. CA Cancer J. Clin., 71, 209-249 (2021).
Suwanwongse & Shabarek, Lithium toxicity in two coronavirus disease 2019 (COVID-19) Patients, Cureus, 12(5), e8384 (May 31, 2020).
Taghipour et al., Evaluation of pre-treatment serum levels of IL-7 and GM-CSF in colorectal cancer patients. Int. J. Mol. Cell Med., 3, 27-34 (2014).
Tavora et al., Glycogen synthase kinase-3β expression in prostate cancer (PCa) correlates with aggressive pathological features and its blockade with 9-ING-41 inhibits viability of PCa cell lines. In: American Association for Cancer Research (AACR)
Annual Meeting 2020, abstract 2959, San Diego: (2020). Experiments in prostate cancer cell lines showed that 9-ING-41 regulated the expression of PD-L1.
Taylor & Rudd, Glycogen synthase kinase 3 (GSK-3) controls T-cell motility and interactions with antigen presenting cells. BMC Research Notes, 13, 163-163 (2020).
Taylor et al., Glycogen synthase kinase 3 inactivation drives T-beta-mediated downregulation of co-receptor PD-1 to enhance CD8(+) cytolytic T cell responses. Immunity, 44, 274-86 (2016). GSK-3 small-molecule inhibitors and GSK-3 siRNA reduced PD-1 expression, increased CD8+ T cell function, and enhanced viral clearance in models of herpes (MHV-68) and lymphocytic choriomeningitis (LCMV-C13) viral infections.
Taylor, Rothstein, & Rudd, Small-molecule inhibition of PD-1 transcription is an effective alternative to antibody blockade in cancer therapy. Cancer Res., 78, 706-17 (2018). The increased T cell function induced by GSK-3 inhibition resulted in anti-tumor activity comparable with the effects observed with anti-PD-1 monoclonal antibodies in animal models of metastatic melanoma and lymphoma.
Ugolkov et al., 9-ING-41, a small molecule glycogen synthase kinase-3 inhibitor, is active in neuroblastoma. Anticancer Drugs, 29(8), 717-724 (September 2018).
Ugolkov et al., Combination treatment with the GSK-3 inhibitor 9-ING-41 and CCNU cures orthotopic chemoresistant glioblastoma in patient-derived xenograft models. Translational Oncology, Volume 10, Issue 4, pages 669-678 (August 2017). -ING-41 is an inhibitor of both the alpha and beta isoform of GSK-3.
Watkins, Egilmez, Suttles, & Stout, IL-12 rapidly alters the functional profile of tumor-associated and tumor-infiltrating macrophages in vitro and in vivo. J. Immunol., 178, 1357-1362 (2007).
Williford et al., Recruitment of CD103(+) dendritic cells via tumor-targeted chemokine delivery enhances efficacy of checkpoint inhibitor immunotherapy. Science advances, 5, eaayl357-eaayl357 (2019).
Wu et al., Targeting glycogen synthase kinase 3 for therapeutic benefit in lymphoma. Blood, 134(4), 363-373 (Jul. 25, 2019).
Yu et al., CXCL12/CXCR4 promotes inflammation-driven colorectal cancer progression through activation of RhoA signaling by sponging miR-133a-3p. Journal of Experimental & Clinical Cancer Research, 38, 32 (2019).
Zhang, Sun, & Feng, [Comparison of clinical and pathological features between severe acute respiratory syndrome and coronavirus disease 2019]. Zhonghua Jie He He Hu Xi Za Zhi. (2020) 43:496-502. Autopsy studies demonstrated marked fibrotic lung disease in patients who suffered from the COVID-19.
Zhong et al., TI PE regulates VEGFR2 expression and promotes angiogenesis in colorectal cancer. Int. J. Biol. Sci., 16, 272-283 (2020).

### TEXTBOOKS AND TECHNICAL REFERENCES

Current Protocols in Immunology (CPI). Coligan et al., eds. (John Wiley and Sons, Inc., 2003).
Current Protocols in Molecular Biology (CPMB). Ausubel, ed. (John Wiley and Sons, 2014).
Current Protocols in Protein Science (CPPS), Coligan, ed. (John Wiley and Sons, Inc., 2005).
Janeway's Immunobiology 9th edition, Murphy et al., eds. (Taylor & Francis Limited, 2017).
Laboratory Methods in Enzymology: DNA. Lorsch, ed. (Elsevier, 2013).
Lewin's Genes XII. Krebs et al., eds. (Jones & Bartlett Publishers (2014).
Luttmann et al., Immunology (Elsevier, 2006).
Molecular Cloning: A Laboratory Manual, 4th edition, Green & Sambrook, eds. (Cold Spring Harbor Laboratory Press, Cold Spring Harbor, N.Y., USA, 2012).
Pharmaceutical Sciences, 23^{rd} edition (Elsevier, 2020).
The Encyclopedia of Molecular Cell Biology and Molecular Medicine, 2^{nd} edition. Meyers, ed. (Wiley-Blackwell, 2004).
The Merck Manual of Diagnosis and Therapy, 19^{th} edition (Merck Sharp & Dohme Corp., 2018).

All patents and publications cited throughout this specification are expressly incorporated by reference to disclose and describe the materials and methods that might be used with the technologies described in this specification. The publications discussed are provided solely for their disclosure before the filing date. They should not be construed as an admission that the inventors may not antedate such disclosure under prior invention or for any other reason. If there is an apparent discrepancy between a previous patent or publication and the description provided in this specification, the present specification (including any definitions) and claims shall control. All statements as to the date or representation as to the contents of these documents are based on the information available to the applicants and constitute no admission as to the correctness of the dates or contents of these documents. The dates of publication provided in this specification may differ from the actual publication dates. If there is an apparent discrepancy between a publication date provided in this specification and the actual publication date supplied by the publisher, the actual publication date shall control.

1. A method of treating colorectal cancer, comprising the step of:
administering a therapeutically effective amount of a GSK-3 inhibitor with structural similarity to 9-ING-41, wherein the GSK-3 inhibitor includes a maleimide group, to a subject with colorectal cancer.

2. The method of claim 1, comprising the step of:
administering a therapeutically effective amount of a compound selected from the group consisting of 9-ING-41, compound CID: 150974278, compound CID: 137495982, compound CID: 59140652; and compound CID: 16741640, to a subject with colorectal cancer.

3. A method of increasing an anti-tumor immune response and decrease tumor burden in a subject, comprising the steps of:
(a) administering a therapeutically effective amount of a GSK-3 inhibitor with structural similarity to 9-ING-41, wherein the GSK-3 inhibitor includes a maleimide group and
(b) administering a therapeutically effective amount of another anti-cancer therapeutic agent.

4. The method of claim 3, wherein the other anti-cancer therapeutic agent is an immune checkpoint therapy.

5. The method of claim 4, wherein the immune checkpoint therapy is a an αPD-1/PD-L1 therapy.

6. A method of treating COVID-19, comprising the step of:
administering a therapeutically effective amount of a GSK-3 inhibitor with structural similarity to 9-ING-41, wherein the GSK-3 inhibitor includes a maleimide group, to a subject with COVID-19.

7. The method of claim 6, comprising the additional step of:
administering a therapeutically effective amount of a second GSK-3 inhibitor, to a subject with COVID-19.

8. The method of claim 7, comprising the additional step of:
administering a therapeutically effective amount of a second GSK-3 inhibitor, to a subject with COVID-19.

9. The method of claim 8, wherein the second GSK-3 inhibitor is lithium.

10. The method of claim 8, comprising the additional step comprises administering the second GSK-3 inhibitor orally or sublingually.

11. The method of claim 6, comprising the additional step of:
monitoring the course of treatment based on inflammatory plasma or serum cytokine profiles.

12. The method of claim 11, comprising the additional step of:
monitoring the course of treatment for lithium-associated toxicity.
